SEQUENCE LISTING (84 sequences)

SEQ ID NO: 1 - protein, 19 aa, Artificial
  Consensus peptide
  MISC_FEATURE (1)..(1): X at position 1 is L or M
  MISC_FEATURE (2)..(2): X at position 2 is any amino acid
  MISC_FEATURE (3)..(3): X at position 3 is any amino acid
  MISC_FEATURE (4)..(4): X at position 4 is L or M
  MISC_FEATURE (5)..(5): X at position 5 is any amino acid
  MISC_FEATURE (6)..(6): X at position 6 is any amino acid
  MISC_FEATURE (8)..(8): X at position 8 is L or M
  MISC_FEATURE (9)..(9): X at position 9 is any amino acid
  MISC_FEATURE (10)..(10): X at position 10 is L or I
  MISC_FEATURE (11)..(11): X at position 11 is L, F, or E
  MISC_FEATURE (12)..(12): X at position 12 is any amino acid
  MISC_FEATURE (13)..(13): X at position 13 is any amino acid
  MISC_FEATURE (14)..(14): X at position 14 is L or I
  MISC_FEATURE (15)..(15): X at position 15 is any amino acid
  MISC_FEATURE (16)..(16): X at position 16 is any amino acid
  MISC_FEATURE (17)..(17): X at position 17 is any amino acid
  MISC_FEATURE (19)..(19): X at position 19 is L or F
XXXXXXLXXXXXXXXXXLX

SEQ ID NO: 2 - protein, 19 aa, Artificial
  Consensus peptide
  MISC_FEATURE (1)..(1): X at position 1 is L or M
  MISC_FEATURE (2)..(2): X at position 2 is any amino acid
  MISC_FEATURE (3)..(3): X at position 3 is any amino acid
  MISC_FEATURE (4)..(4): X at position 4 is L or M
  MISC_FEATURE (6)..(6): X at position 6 is E or Q
  MISC_FEATURE (8)..(8): X at position 8 is L or M
  MISC_FEATURE (9)..(9): X at position 9 is any amino acid
  MISC_FEATURE (10)..(10): X at position 10 is L or I
  MISC_FEATURE (11)..(11): X at position 11 is L, F, or E
  MISC_FEATURE (12)..(12): X at position 12 is any amino acid
  MISC_FEATURE (13)..(13): X at position 13 is any amino acid
  MISC_FEATURE (14)..(14): X at position 14 is L or I
  MISC_FEATURE (15)..(15): X at position 15 is any amino acid
  MISC_FEATURE (16)..(16): X at position 16 is E or Q
  MISC_FEATURE (17)..(17): X at position 17 is any amino acid
  MISC_FEATURE (19)..(19): X at position 19 is L or F
XXXXEXLXXXXXXXXXXLX

SEQ ID NO: 3 - protein, 19 aa, Artificial
  Consensus peptide
  MISC_FEATURE (1)..(1): X at position 1 is L or M
  MISC_FEATURE (2)..(2): X at position 2 is R, K, D, E, Q, N, H, S, T, G, P, Y, W, or A
  MISC_FEATURE (3)..(3): X at position 3 is R, K, D, E, Q, N, H, S, T, G, P, Y, W, or A
  MISC_FEATURE (4)..(4): X at position 4 is L or M
  MISC_FEATURE (6)..(6): X at position 6 is R, K, D, E, Q, N, H, S, T, G, P, Y, W, or A
  MISC_FEATURE (8)..(8): X at position 8 is L or M
  MISC_FEATURE (9)..(9): X at position 9 is R, K, D, E, Q, N, H, S, T, G, P, Y, W, or A
  MISC_FEATURE (12)..(12): X at position 12 is R, K, D, E, Q, N, H, S, T, G, P, Y, W, or A
  MISC_FEATURE (13)..(13): X at position 13 is R, K, D, E, Q, N, H, S, T, G, P, Y, W, or A
  MISC_FEATURE (15)..(15): X at position 15 is R, K, D, E, Q, N, H, S, T, G, P, Y, W, or A
  MISC_FEATURE (16)..(16): X at position 16 is R, K, D, E, Q, N, H, S, T, G, P, Y, W, or A
  MISC_FEATURE (17)..(17): X at position 17 is R, K, D, E, Q, N, H, S, T, G, P, Y, W, or A
XXXXEXLXXIFXXIXXXLF

SEQ ID NO: 4 - protein, 22 aa, Artificial
  Consensus peptide
  MISC_FEATURE (4)..(4): X at position 4 is L or M
  MISC_FEATURE (7)..(7): X at position 7 is L or M
  MISC_FEATURE (11)..(11): X at position 11 is L or M
TSGXSPXEQLXKIFADITQSLF

SEQ ID NO: 5 - protein, 39 aa, Artificial
  P12 peptide
QTGDDSLSGAGQTSGMSPMEQLMKIFADITQSLFGDQDG

SEQ ID NO: 6 - protein, 39 aa, Artificial
  P12-2 peptide
GDLQGSGASTQDTSGMSPMEQLMKIFADITQSLFGDQDG

SEQ ID NO: 7 - protein, 23 aa, Artificial
  P13 peptide
TSGMSPMEQLMKIFADITQSLFG

SEQ ID NO: 8 - protein, 23 aa, Artificial
  P13-2 peptide
TSGLSPLEQLLKIFADITQSLFG

SEQ ID NO: 9 - protein, 23 aa, Artificial
  P13-3 peptide
TSGLSPLEQLLKIFAEITQSLFG

SEQ ID NO: 10 - protein, 23 aa, Artificial
  P13-4 peptide
MSPMEQLMKIFADITQSLFEEEE

SEQ ID NO: 11 - protein, 23 aa, Artificial
  P13-5 peptide
LSPLEQLMKIFADITQSLFEEEE

SEQ ID NO: 12 - protein, 21 aa, Artificial
  P13-6 peptide
MEEMEELMEIFEEIEEELFEE

SEQ ID NO: 13 - protein, 21 aa, Artificial
  P13-7 peptide
LEELEELLEIFEEIEEELFEE

SEQ ID NO: 14 - protein, 24 aa, Artificial
  P13-8 peptide
SEEEEMSPMEQLMKIFADITQSLF

SEQ ID NO: 15 - protein, 24 aa, Artificial
  P13-9 peptide
SEEEEMSPMEQLMKIFAEITQSLF

SEQ ID NO: 16 - protein, 36 aa, Artificial
  P13-10 peptide
DDSLSGAGQTSGMSPMEQLMKIFADITQSLFGDQDG

SEQ ID NO: 17 - protein, 33 aa, Artificial
  P13-11 peptide
LSGAGQTSGMSPMEQLMKIFADITQSLFGDQDG

SEQ ID NO: 18 - protein, 30 aa, Artificial
  P13-12 peptide
AGQTSGMSPMEQLMKIFADITQSLFGDQDG

SEQ ID NO: 19 - protein, 37 aa, Artificial
  P13-13 peptide
QTGDDSLSGAGQTSGMSPMEQLMKIFADITQSLFGDQ

SEQ ID NO: 20 - protein, 35 aa, Artificial
  P13-14 peptide
QTGDDSLSGAGQTSGMSPMEQLMKIFADITQSLFG

SEQ ID NO: 21 - protein, 25 aa, Artificial
  P13-15 peptide
GQTSGMSPMEQLMKIFADITQSLFG

SEQ ID NO: 22 - protein, 26 aa, Artificial
  P13-16 peptide
AGQTSGMSPMEQLMKIFADITQSLFG

SEQ ID NO: 23 - protein, 28 aa, Artificial
  P13-17 peptide
AGQTSGMSPMEQLMKIFADITQSLFGDQ

SEQ ID NO: 24 - protein, 30 aa, Artificial
  P13-18 peptide
AGQTSGMSPMEQLMEIFADITQSLFGDQDG

SEQ ID NO: 25 - protein, 30 aa, Artificial
  P13-19 peptide
AGQTSGMSPMEQLMAIFADITQSLFGDQDG

SEQ ID NO: 26 - protein, 31 aa, Artificial
  P13-20 peptide
AGQTSGMSPMEQLMEIFADITQSLFGDQDGR

SEQ ID NO: 27 - protein, 31 aa, Artificial
  P13-21 peptide
AGQTSGMSPMEQLMAIFADITQSLFGDQDGR

SEQ ID NO: 28 - protein, 31 aa, Artificial
  P13-22 peptide
AGQTSGMSPMEQLMEIFADITQSLFGDQDGK

SEQ ID NO: 29 - protein, 26 aa, Artificial
  P13-23 peptide
AGQTSGLSPLEQLLKIFADITQSLFG

SEQ ID NO: 30 - protein, 24 aa, Artificial
  P13-24 peptide
GQTSGMSPMEQLMEIFADITQSLF

SEQ ID NO: 31 - protein, 24 aa, Artificial
  P13-25 peptide
SQTSGMSPMEQLMEIFADITQSLF

SEQ ID NO: 32 - protein, 25 aa, Artificial
  P13-26 peptide
SQEEEMEPMEQLMEIFEEIEQELFG

SEQ ID NO: 33 - protein, 25 aa, Artificial
  P13-27 peptide
SQEEEMEEMEQLMEIFEEIEQELFG

SEQ ID NO: 34 - protein, 26 aa, Artificial
  P13-28 peptide
SEQEEEMEEMEQLMEIFEEIEQELFE

SEQ ID NO: 35 - protein, 26 aa, Artificial
  P13-29 peptide
SEQEEELEELEQLLEIFEEIEQELFE

SEQ ID NO: 36 - protein, 30 aa, Artificial
  P13-30 peptide
AGQTSGMSPMEQLMKLFADLTQSLFGDQDG

SEQ ID NO: 37 - protein, 30 aa, Artificial
  P13-31 peptide
AGQTSGMSPMEQLMKILADITQSLFGDQDG

SEQ ID NO: 38 - protein, 30 aa, Artificial
  P13-32 peptide
AGQTSGMSPMEQLMKIFADITQSLLGDQDG

SEQ ID NO: 39 - protein, 30 aa, Artificial
  P13-33 peptide
AGQTSGMSPMEQLMKILADITQSLLGDQDG

SEQ ID NO: 40 - protein, 25 aa, Artificial
  P13-34 peptide
SEQEEEMEEMEQLMEIFEEIEQELF

SEQ ID NO: 41 - protein, 19 aa, Artificial
  P13-35 peptide
LEELEELLEIFEEIEEELF

SEQ ID NO: 42 - protein, 24 aa, Artificial
  P13-36 peptide
SEEMSPMEQLMKIFADITQSLFEE

SEQ ID NO: 43 - protein, 23 aa, Artificial
  P13-37 peptide
MEEMEQLMKIFEEIEQELFEEEE

SEQ ID NO: 44 - protein, 23 aa, Artificial
  P13-38 peptide
MSPMEELMKIFADITESLFEEEE

SEQ ID NO: 45 - protein, 21 aa, Artificial
  P13-s5 peptide
MSPMEQLMKIFADITQSLFEE

SEQ ID NO: 46 - protein, 23 aa, Artificial
  P13-s6 peptide
MEEMEQLMEIFEEIEQELFEEEE

SEQ ID NO: 47 - protein, 23 aa, Artificial
  P13-s7 peptide
MSPMEQLMEIFADITQSLFEEEE

SEQ ID NO: 48 - protein, 21 aa, Artificial
  P13-s8 peptide
LEEMEELMEIFEEIEEELFEE

SEQ ID NO: 49 - protein, 21 aa, Artificial
  P13-s9 peptide
MEELEELMEIFEEIEEELFEE

SEQ ID NO: 50 - protein, 21 aa, Artificial
  P13-s10 peptide
MEEMEELLEIFEEIEEELFEE

SEQ ID NO: 51 - protein, 21 aa, Artificial
  P13-s11 peptide
MEELEELLEIFEEIEEELFEE

SEQ ID NO: 52 - protein, 21 aa, Artificial
  P13-s12 peptide
LEEMEELLEIFEEIEEELFEE

SEQ ID NO: 53 - protein, 21 aa, Artificial
  P13-s13 peptide
LEELEELMEIFEEIEEELFEE

SEQ ID NO: 54 - protein, 5 aa, Artificial
  enterokinase specific cleavage site
DDDDK

SEQ ID NO: 55 - protein, 4 aa, Artificial
  factor Xa specific cleavage site
  MISC_FEATURE (2)..(2): X at position 2 is E or D
IXGR

SEQ ID NO: 56 - protein, 6 aa, Artificial
  GenenaseT I specific cleavage site
PGAAHY

SEQ ID NO: 57 - DNA, 117 bp, Artificial
  P12 in E. coli
cagaccggtgatgatagcctgagcggtgcaggtcagaccagcggtatgagcccgatggaacagctgatgaaaatttttgcagatattacccagagcctgtttggtgatcaggatggt

SEQ ID NO: 58 - DNA, 93 bp, Artificial
  P13-20 in E. coli
gcaggtcagaccagcggtatgagcccgatggaacagctgatggaaatttttgcagatattacccagagcctgtttggtgatcaggatggtcgt

SEQ ID NO: 59 - DNA, 117 bp, Artificial
  P12 in Zea mays
cagaccggcgacgactccctgtccggcgccggccagacctccggcatgtccccgatggagcagctgatgaagatcttcgccgacatcacccagtccctgttcggcgaccaggacggc

SEQ ID NO: 60 - DNA, 93 bp, Artificial
  P13-20 in Zea mays
gccggccagacctccggcatgtccccgatggagcagctgatggagatcttcgccgacatcacccagtccctgttcggcgaccaggacggcagg

SEQ ID NO: 61 - protein, 30 aa, Artificial
  P13-39 peptide
AGQTSGMSPMEQLLKIFADITQSLFGDQDG

SEQ ID NO: 62 - protein, 30 aa, Artificial
  P13-40 peptide
AGQTSGMSPLEQLMKIFADITQSLFGDQDG

SEQ ID NO: 63 - protein, 30 aa, Artificial
  P13-41 peptide
AGQTSGLSPMEQLMKIFADITQSLFGDQDG

SEQ ID NO: 64 - protein, 30 aa, Artificial
  P13-42 peptide
AGETSGMSPMEQLMKIFADITQSLFGDQDG

SEQ ID NO: 65 - protein, 30 aa, Artificial
  P13-43 peptide
AGQTSGMSPMEQLMKIFADITESLFGDQDG

SEQ ID NO: 66 - protein, 30 aa, Artificial
  P13-44 peptide
AGQTSGMSPMEQLMKIFADITQSLFGDEDG

SEQ ID NO: 67 - protein, 30 aa, Artificial
  P13-45 peptide
AGQTSGMSPMEELMKIFADITQSLFGDQDG

SEQ ID NO: 68 - protein, 30 aa, Artificial
  P13-46 peptide
AEQEEEMEPMEQLMKIFEEIEQELFEEEEE

SEQ ID NO: 69 - protein, 30 aa, Artificial
  P13-47 peptide
AGQTSGESPMEQLMKIFADITQSLFGDQDG

SEQ ID NO: 70 - protein, 30 aa, Artificial
  P13-48 peptide
AGQTSGMSPEEQLMKIFADITQSLFGDQDG

SEQ ID NO: 71 - protein, 30 aa, Artificial
  P13-49 peptide
AGQTSGMSPMEQEMKIFADITQSLFGDQDG

SEQ ID NO: 72 - protein, 30 aa, Artificial
  P13-50 peptide
AGQTSGMSPMEQLEKIFADITQSLFGDQDG

SEQ ID NO: 73 - protein, 30 aa, Artificial
  P13-51 peptide
AGQTSGMSPMEQLMKEFADITQSLFGDQDG

SEQ ID NO: 74 - protein, 30 aa, Artificial
  P13-52 peptide
AGQTSGMSPMEQLMKIEADITQSLFGDQDG

SEQ ID NO: 75 - protein, 30 aa, Artificial
  P13-53 peptide
AGQTSGMSPMEQLMKIFADETQSLFGDQDG

SEQ ID NO: 76 - protein, 30 aa, Artificial
  P13-54 peptide
AGQTSGMSPMEQLMKIFADITQSEFGDQDG

SEQ ID NO: 77 - protein, 30 aa, Artificial
  P13-55 peptide
AGQTSGMSPMEQLMKIFADITQSLEGDQDG

SEQ ID NO: 78 - protein, 30 aa, Artificial
  P13-56 peptide
AGQTSGMSPMEQLMEIFADITQSLFGDQDR

SEQ ID NO: 79 - protein, 29 aa, Artificial
  P13-57 peptide
AGQTSGMSPMEQLMEIFADITQSLFGDQR

SEQ ID NO: 80 - protein, 28 aa, Artificial
  P13-58 peptide
AGQTSGMSPMEQLMEIFADITQSLFGDR

SEQ ID NO: 81 - protein, 5 aa, Artificial
  Peptide
SEEEE

SEQ ID NO: 82 - protein, 4 aa, Artificial
  Peptide
EEEE

SEQ ID NO: 83 - protein, 24 aa, Artificial
  P13-s14 peptide
TSGLSPLEQLLEIFADITQSLFGR

SEQ ID NO: 84 - protein, 24 aa, Artificial
  P13-s15 peptide
TSGLSPLEQLLEIFAEITQSLFGR

(19) United States
(12) Patent Application Publication
(10) Pub. No.: US 2018/0362993 A1
(43) Pub. Date: Dec. 20, 2018

(54) ELICITOR-DERIVED PEPTIDES AND USE THEREOF

(71) Applicant: Plant Health Care, Inc., Raleigh, NC (US)

(72) Inventors: Zhongmin Wei, Kirkland, WA (US); Gregory A. Zornetzer, Seattle, WA (US)

(21) Appl. No.: 15/989,647

(22) Filed: May 25, 2018

Related U.S. Application Data
(60) Provisional application No. 62/511,517, filed on May 26, 2017.

(51) Int. Cl. C12N 15/82; A01N 51/00; C07C 271/04

(52) U.S. Cl. CPC C12N 15/8202; C12N 15/8273; C07C 271/04; A01N 51/00; C12N 15/8285; C12N 15/8282

(57) ABSTRACT

Disclosed are non-hypersensitive response eliciting peptides and weak hypersensitive response eliciting peptides that induce active plant responses, and that exhibit improved solubility, stability, resistance to chemical degradation, or a combination of these properties. Use of these peptides or fusion polypeptides, compositions, recombinant host cells or DNA constructs encoding the same, for modulating plant biochemical signaling, imparting disease resistance to plants, enhancing plant growth, imparting tolerance to biotic stress, imparting tolerance and resistance to abiotic stress, imparting desiccation resistance to cuttings removed from ornamental plants, imparting post-harvest disease or post-harvest desiccation resistance to a fruit or vegetable, or enhancing the longevity of fruit or vegetable ripeness are also disclosed.

This application claims the priority benefit of U.S. Provisional Patent Application Ser. No. 62/511,517, filed May 26, 2017, which is hereby incorporated by reference in its entirety.

### FIELD OF THE INVENTION

The present invention relates to novel hypersensitive response elicitor peptides and their use for inducing active plant responses including, among others, growth enhancement, disease resistance, pest or insect resistance, and stress resistance.

### BACKGROUND OF THE INVENTION

The identification and isolation of harpin proteins came from basic research at Cornell University attempting to understand how plant pathogenic bacteria interact with plants. A first line of defense is the hypersensitive response (HR), a localized plant cell death at the site of infection. Cell death creates a physical barrier to movement of the pathogen and in some plants dead cells can release compounds toxic to the invading pathogen. Research had indicated that pathogenic bacteria were likely to have a single factor that was responsible for triggering the HR. A basic aim of the Cornell research was to identify a specific bacterial protein responsible for eliciting the HR. The target protein was known to be encoded by one of a group of bacteria genes called the Hypersensitive Response and Pathogenicity (hrp) gene cluster. The hrp cluster in the bacterium Envinia amylovora (Ea), which causes fire blight in pear and apple, was dissected and a single protein was identified that elicited HR in certain plants. This protein was given the name harpin (and, later, harpin_{Eo} and the corresponding gene designated hrpN. This was the first example of such a protein and gene identified from any bacterial species.

A number of different harpin proteins have since been identified from Envinia, Pseudomonas, Ralstonia, Xanthomonas, and Pantoea species, among others. Harpin proteins, while diverse at the primary amino acid sequence level, share common biochemical and biophysical characteristics as well as biological functions. Based on their unique properties, the harpin proteins are regarded in the literature as belonging to a single class of proteins.

Subsequent to their identification and isolation, it was thereafter discovered that harpins could elicit disease resistance in plants and increase plant growth. An important early finding was that application of purified harpin protein made a plant resistant to a subsequent pathogen attack, and in locations on the plant well away from the injection site. This meant that harpin proteins can trigger a Systemic Acquired Resistance (SAR), a plant defense mechanism that provides resistance to a variety of viral, bacterial, and fungal pathogens.

In crop protection, there is a continuous need for compositions that improve the health of plants. Healthier plants are desirable since they result in better yields and/or a better quality of the plants or crops. Healthier plants also better resist biotic and abiotic stress. A high resistance against biotic stresses in turn allows the growers to reduce the quantity of pesticides applied and consequently to slow down the development of resistances against the respective pesticides.

Harpin_{αβ}, is a fusion protein that is derived from several different harpins. HarHarpin_{αβ}, has been shown to suppress nematode egg production, enhance the growth, quality and yield of a plant, and increase a plant's vigor. Its amino acid and nucleotide sequences are described in detail in U.S. Application Publ. No. 2010/0043095.

To date, harpin and harpin_{αβ}, production and their use in agricultural and horticultural applications have been as a powdered solid coated on starch. This limits the use and versatility of the harpin proteins, because liquid suspensions of the powdered harpin proteins in water have an effective useful life of only 48-72 hours before significant degradation and loss of activity occurs. Another problem with harpin solutions is protein solubility and stability.

It would be desirable to identify synthetic and derivative harpin peptides that are readily soluble in aqueous solution, stable, resistant to chemical degradation, and effective in initiating one or more active plant responses including, without limitation, disease and/or drought resistance.

The present invention is directed to overcoming these and other limitations in the art.

### SUMMARY OF THE INVENTION

A first aspect of the invention relates to an isolated peptide comprising the amino acid sequence of

(SEQ ID NO: 1)
(L/M)-X-X-(L/M)-X-X-L-(L/M)-X-(L/I)-(E/L/F)-X-X-
(L/I)-X-X-X-L-(L/F)

wherein each X is independently any amino acid.

A second aspect of the invention relates to an isolated peptide according to the first aspect of the present invention, wherein the peptide comprises the amino acid sequence of

(SEQ ID NO: 2)
(L/M)-X-X-(L/M)-E-(E/Q)-L-(L/M)-X-(L/I)-(E/L/F)-X-
X-(L/I)-X-(E/Q)-X-L-(L/F),

wherein each X is independently any amino acid.

A third aspect of the present invention relates to an isolated peptide according to the first aspect of the present invention, wherein the peptide comprises the amino acid sequence of: (L/M)-X-X-(L/M)-E-X-L-(L/M)-X-I-F-X-X-I-X-X-X-L-F (SEQ ID NO:3), wherein each X is independently one of R, K, D, E, Q, N, H, S, T, G, P, Y, W, or A.

A fourth aspect of the present invention relates to an isolated peptide according to the first aspect of the present invention, wherein the peptide comprises the amino acid sequence of:

(SEQ ID NO: 4)
T-S-G-(L/M)-S-P-(L/M)-E-Q-L-(L/M)-K-I-F-A-D-I-T-Q-
S-L-F.

A fifth aspect of the invention relates to a fusion polypeptide that includes one of the peptides of the first, second, third, or fourth aspect of the invention along with one or more of a purification tag, a solubility tag, or a second peptide according to the first or second aspect of the invention.

A sixth aspect of the invention relates to a composition that includes one or more peptides according to the first, second, third, or fourth aspect of the invention, or a fusion polypeptide according to the fifth aspect of the invention, and a carrier.

A seventh aspect of the invention relates to a recombinant host cell comprising a transgene that comprises a promoter-effective nucleic acid molecule operably coupled to a nucleic acid molecule that encodes a peptide or fusion polypeptide according to the first, second, third, fourth, or fifth aspect of the invention, respectively, wherein the recombinant host cell is a microbe that imparts a first benefit to a plant grown in the presence of the recombinant microbe and the peptide or fusion polypeptide imparts a second benefit to the plant grown in the presence of the recombinant microbe.

An eight aspect of the invention relates to a method of imparting disease resistance to plants. This method includes: applying an effective amount of an isolated peptide according to the first, second, third, or fourth aspect of the invention, a fusion polypeptide according to the fifth aspect of the invention, a composition according to the sixth aspect of the invention, or a recombinant host cell according to the seventh aspect of the invention to a plant or plant seed or the locus where the plant is growing or is expected to grow, wherein said applying is effective to impart disease resistance.

A ninth aspect of the invention relates to a method of enhancing plant growth. This method includes: applying an effective amount of an isolated peptide according to the first, second, third, or fourth aspect of the invention, a fusion polypeptide according to the fifth aspect of the invention, a composition according to the sixth aspect of the invention, or a recombinant host cell according to the seventh aspect of the invention to a plant or plant seed or the locus where the plant is growing or is expected to grow, wherein said applying is effective to enhance plant growth.

A tenth aspect of the invention relates to a method of increasing a plant's tolerance and resistance to biotic stressors. This method includes: applying an effective amount of an isolated peptide according to the first, second, third, or fourth aspect of the invention, a fusion polypeptide according to the fifth aspect of the invention, a composition according to the sixth aspect of the invention, or a recombinant host cell according to the seventh aspect of the invention to a plant or plant seed or the locus where the plant is growing or is expected to grow, wherein said applying is effective to increase the plant's tolerance and resistance to biotic stress factors selected from the group consisting of pests such as insects, arachnids, nematodes, weeds, and combinations thereof.

An eleventh aspect of the invention relates to a method of increasing a plant's tolerance to abiotic stress. This method includes: applying an effective amount of an isolated peptide according to the first, second, third, or fourth aspect of the invention, a fusion polypeptide according to the fifth aspect of the invention, a composition according to the sixth aspect of the invention, or a recombinant host cell according to the seventh aspect of the invention to a plant or plant seed or the locus where the plant is growing or is expected to grow, wherein said applying is effective to increase the plant's tolerance to abiotic stress factors selected from the group consisting of salt stress, water stress (including drought and flooding), ozone stress, heavy metal stress, cold stress, heat stress, nutritional stress (phosphate, potassium, nitrogen deficiency), bleaching and light-induced stress, and combinations thereof.

A twelfth aspect of the invention relates to a method imparting desiccation resistance to cuttings removed from ornamental plants. This method includes: applying an isolated peptide according to the first, second, third, or fourth aspect of the invention, a fusion polypeptide according to the fifth aspect of the invention, a composition according to the sixth aspect of the invention, or a recombinant host cell according to the seventh aspect of the invention to a plant or the locus where the plant is growing, wherein said applying is effective to impart desiccation resistance to cuttings removed from the ornamental plant.

A thirteenth aspect of the invention relates to a method of imparting post-harvest disease or post-harvest desiccation resistance to a fruit or vegetable. This method includes: applying an effective amount of an isolated peptide according to the first, second, third, or fourth aspect of the invention, a fusion polypeptide according to the fifth aspect of the invention, a composition according to the sixth aspect of the invention, or a recombinant host cell according to the seventh aspect of the invention to a plant containing a fruit or vegetable or the locus where the plant is growing; or applying an effective amount of the isolated peptide, the fusion polypeptide, or the composition to a harvested fruit or vegetable, wherein said applying is effective to impart post-harvest disease resistance or desiccation resistance to the fruit or vegetable.

A fourteenth aspect of the invention relates to a method of enhancing the longevity of fruit or vegetable ripeness. This method includes: applying an effective amount of an isolated peptide according to the first, second, third, or fourth aspect of the invention, a fusion polypeptide according to the fifth aspect of the invention, a composition according to the sixth aspect of the invention, or a recombinant host cell according to the seventh aspect of the invention to a plant containing a fruit or vegetable or the locus where the plant is growing; or applying an effective amount of the isolated peptide, the fusion polypeptide, or the composition to a harvested fruit or vegetable, wherein said applying is effective to enhance the longevity of fruit or vegetable ripeness.

A fifteenth aspect of the invention relates to a method of modulating one or more biological signaling processes of a plant. This method includes: applying an effective amount of an isolated peptide according to the first, second, third, or fourth aspect of the invention, a fusion polypeptide according to the fifth aspect of the invention, a composition according to the sixth aspect of the invention, or a recombinant host cell according to the seventh aspect of the invention to a plant or the locus where the plant is growing, wherein said applying is effective in modulating one or more biochemical signaling processes.

A sixteenth aspect of the invention relates to a method of treating plant seeds. This method includes providing one or more plant seeds and applying to the provided one or more plant seeds either a recombinant host cell according to the seventh aspect of the invention or a composition according to the sixth aspect of the invention.

A seventeenth aspect of the invention relates to a method of treating plants. This method includes providing one or more plants and applying to the provided one or more plants either a recombinant host cell according to the seventh aspect of the invention or a composition according to the sixth aspect of the invention.

An eighteenth aspect of the invention relates to a method for treating plants. This methods includes applying to a locus where plants are being grown or are expected to be grown either a recombinant host cell according to the seventh aspect of the invention or a composition according to the sixth aspect of the invention, and growing one or more plants at the locus where the recombinant host cell or the composition is applied.

A nineteenth aspect of the invention relates to a DNA construct including a first nucleic acid molecule encoding a peptide according to the first, second, third, or fourth aspect of the invention or a fusion polypeptide according to the fifth aspect of the invention; and a promoter-effective nucleic acid molecule operably coupled to the first nucleic acid molecule. This aspect of the invention also encompasses a recombinant expression vector containing the DNA construct, a recombinant host cell containing the DNA construct, as well as transgenic plants or plant seeds that include a recombinant plant cell of the invention (which contains the DNA construct).

A twentieth aspect of the invention relates to a method of imparting disease resistance to plants, enhancing plant growth, imparting tolerance and resistance to biotic stressors, imparting tolerance to abiotic stress, or modulating plant biochemical signaling. This method includes providing a transgenic plant transformed with a DNA construct according to the nineteenth aspect of the invention; and growing the plant under conditions effective to permit the DNA construct to express the peptide or the fusion polypeptide to impart disease resistance, enhance plant growth, impart tolerance to biotic stress, impart tolerance to abiotic stress, or modulate biochemical signaling to the transgenic plant.

A twenty-first aspect of the invention relates to a method of imparting desiccation resistance to cuttings removed from ornamental plants, imparting post-harvest disease or post-harvest desiccation resistance to a fruit or vegetable, or enhancing the longevity of fruit or vegetable ripeness. The method includes providing a transgenic plant transformed with a DNA construct providing a transgenic plant transformed with a DNA construct according to the nineteenth aspect of the invention; and growing the plant under conditions effective to permit the DNA construct to express the peptide or the fusion polypeptide to impart desiccation resistance to cuttings removed from a transgenic ornamental plant, impart post-harvest disease resistance or desiccation resistance to a fruit or vegetable removed from the transgenic plant, or enhance longevity of ripeness for a fruit or vegetable removed from the transgenic plant.

A twenty-second aspect of the invention relates to a method of imparting disease resistance to plants, enhancing plant growth, imparting tolerance and resistance to biotic stressors, imparting tolerance to abiotic stress, or modulating biochemical signaling. This method includes providing a transgenic plant seed transformed with a DNA construct according to the nineteenth aspect of the invention; planting the transgenic plant seed in soil; and propagating a transgenic plant from the transgenic plant seed to permit the DNA construct to express the peptide or the fusion polypeptide to impart disease resistance, enhance plant growth, impart tolerance to biotic stress, or impart tolerance to abiotic stress to the transgenic plant.

A twenty-third aspect of the invention relates to a method of imparting desiccation resistance to cuttings removed from ornamental plants, imparting post-harvest disease or post-harvest desiccation resistance to a fruit or vegetable, or enhancing the longevity of fruit or vegetable ripeness. The method includes providing a transgenic plant seed transformed with a DNA construct according to the nineteenth aspect of the invention; planting the transgenic plant seed in soil; and propagating a transgenic plant from the transgenic plant seed to permit the DNA construct to express the peptide or the fusion polypeptide to impart desiccation resistance to cuttings removed from a transgenic ornamental plant, impart post-harvest disease resistance or desiccation resistance to a fruit or vegetable removed from the transgenic plant, or enhance longevity of ripeness for a fruit or vegetable removed from the transgenic plant.

By providing active but non-HR-eliciting/weak HR-eliciting peptides that exhibit improved solubility, stability, resistance to chemical degradation, or a combination of these properties, it will afford growers with greater flexibility in preparing, handling, and delivering to plants in their fields or greenhouses effective amounts of compositions containing these non-HR-eliciting/weak HR-eliciting peptides. Simplifying the application process for growers will lead to greater compliance and, thus, improved results with respect to one or more of disease resistance, growth enhancement, tolerance and resistance to biotic stressors, tolerance to abiotic stress, desiccation resistance for cuttings removed from ornamental plants, post-harvest disease resistance or desiccation resistance to fruit or vegetables harvested from plants, and/or improved longevity of fruit or vegetable ripeness for fruit or vegetables harvested from plants. These and other benefits are described herein.

### DETAILED DESCRIPTION OF THE INVENTION

One aspect of the invention relates to novel peptides that possess the ability to promote active plant responses (which may or may not include a hypersensitive response) that afford one or more of the following attributes: disease resistance, growth enhancement, tolerance and resistance to biotic stressors, tolerance to abiotic stress, desiccation resistance for cuttings removed from ornamental plants, post-harvest disease resistance or desiccation resistance to fruit or vegetables harvested from plants, and/or improved longevity of fruit or vegetable ripeness for fruit or vegetables harvested from plants. The induction of these plant responses involves modulating plant biochemical signaling.

As used herein, naturally occurring amino acids are identified throughout by the conventional three-letter and/or one-letter abbreviations, corresponding to the trivial name of the amino acid, in accordance with the following list: Alanine (Ala, A), Arginine (Arg, R), Asparagine (Asn, N), Aspartic acid (Asp, D), Cysteine (Cys, C), Glutamic acid (Glu, E), Glutamine (Gln, Q), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). The abbreviations are accepted in the peptide art and are recommended by the IUPAC-IUB commission in biochemical nomenclature. Naturally occurring variations of amino acids include, without limitation, gamma-glutamate (g-Glu) and isoaspartate (iso-Asp or isoD).

The term “amino acid” further includes analogues, derivatives, and congeners of any specific amino acid referred to herein, as well as C-terminal or N-terminal protected amino acid derivatives (e.g., modified with an N-terminal , C-terminal, or side-chain protecting group, including but not limited to acetylation, formylation, methylation, amidation, esterification, PEGylation, and addition of lipids. Non-naturally occurring amino acids are well known and can be introduced into peptides of the present invention using solid phase synthesis as described below. Furthermore, the term “amino acid” includes both D- and L-amino acids. Hence, an amino acid which is identified herein by its name, three letter or one letter symbol and is not identified specifically as having the D or L configuration, is understood to assume any one of the D or L configurations. In one embodiment, a peptide comprises all L-amino acids.

In certain embodiments, peptides are identified to “consist of” a recited sequence, in which case the peptide includes only the recited amino acid sequence(s) without any extraneous amino acids at the N- or C-terminal ends thereof. To the extent that a recited sequence is in the form of a consensus sequence where one or more of the denoted X or Xaa residues can be any of one or more amino acids, then multiple peptide sequences are embraced by a peptide consisting of such a recited sequence.

In certain other embodiments, peptides are identified to “consist essentially of” a recited sequence, in which case the peptide includes the recited amino acid sequence(s) optionally with one or more extraneous amino acids at the N- and/or C-terminal ends thereof, which extraneous amino acids do not materially alter one or more of the following properties: (i) the ability of the peptide to induce an active response in plants, (ii) solubility of the peptide in water or aqueous solutions, (iii) stability of the peptide dissolved in water or aqueous solution at 50° C. over a period of time (e.g., 3 weeks), and (iv) resistance of the peptide to chemical degradation in the presence of an aqueous buffered solution that includes a biocidal agent (e.g., Proxel®GXL) at 50° C. over a period of time (e.g., 3 weeks).

Briefly, the stability and resistance to chemical degradation of peptides can be assessed as follows using peptide samples having an initial purity of at least about 80%, at least about 82%, at least about 84%, at least about 86%, at least about 88%, at least about 90%, at least about 92%, at least about 94%, at least about 96%, or at least about 98%. For water stability, the peptide is dissolved directly in de-ionized water. For chemical degradation tests, the peptide is dissolved in an aqueous solution containing 50 mM pH buffer and 0.25% Proxel GXL. Exemplary pH buffers include, without limitation: (i) Citrate pH 5.6; (ii) MES pH 6.0; (iii) MOPS pH 6.5; (iv) imidazole pH 7.5; (v) Citrate pH 7.2; (vi) EDDS, pH 7.3; (vii) EDTA pH 8.0; (viii) sodium phosphate pH 8.0; or (ix) TES pH 8.0. Peptides are first dissolved in the aqueous solution at a concentration of 0.5 mg/ml. The samples are incubated at 50° C. to allow for accelerated degradation. An initial sample of the peptide is removed, diluted 10× with water, and analyzed by reverse-phase HPLC. Briefly, 20 μl of the sample is injected into the solvent flow of an HPLC instrument and analyzed on a C18 HPLC column (YMC ProPack C18, YMC, Japan, or C18 Stablebond, Agilent Technologies, USA) using either a triethylamine phosphate in water/acetonitrile gradient or a 0.1% TFA in water/0.1% TFA in acetonitrile gradient to separate different peptide species. Eluting peptides are monitored by UV absorbance at 218 nm and quantified based on the area under the peak. The area under the peak for the initial peptide sample is treated as the standard for relative quantification in subsequent runs. At regular intervals (e.g., 1, 3, 7, 10, and 14 days), each peptide sample is surveyed and analyzed by HPLC as described above. If necessary to observe degradation (i.e., where the peptide exhibits a high degree of chemical stability), this protocol can be extended by several weeks to observe degradation. The quantification of subsequent peptide runs is expressed as a percentage of the original (day 0) HPLC result.

A peptide that is at least partially soluble in water or aqueous solution exhibits a solubility of greater than 0.1 mg/ml, preferably at least about 1.0 mg/ml, at least about 2.0 mg/ml, at least about 3.0 mg/ml, or at least about 4.0 mg/ml. In certain embodiments, the peptide exhibits high solubility in water or aqueous solution, with a solubility of at least about 5.0 mg/ml, at least about 10.0 mg/ml, at least about 15.0 mg/ml, or at least about 20 mg/ml.

A peptide that is stable in water or aqueous solution exhibits at least about 66%, at least about 68%, at least about 70%, at least about 72%, at least about 74%, at least about 76%, at least about 78%, at least about 80%, at least about 82%, at least about 84%, at least about 86%, at least about 88%, or at least about 90% of the original peptide concentration over the designated period of time incubated at 50° C. In certain embodiments, the designated period of time is 3 days, 7 days, 14 days, 21 days, 28 days, one month, two months, three months, or four months.

A peptide that is resistant to chemical degradation exhibits at least about 66%, at least about 68%, at least about 70%, at least about 72%, at least about 74%, at least about 76%, at least about 78%, at least about 80%, at least about 82%, at least about 84%, at least about 86%, at least about 88%, or at least about 90% of the original peptide concentration over the designated period of time incubated at 50° C. In certain embodiments, the designated period of time is 3 days, 7 days, 14 days, 21 days, 28 days, one month, two months, three months, or four months.

A property of a peptide to elicit a hypersensitive response, or not, upon infiltration or application of the peptide to plant tissues can be measured by applying the peptide in dry powder form or in solution form to a plant, particularly though not exclusively a plant leaf. Application rates include 1-500 μg/ml for liquid solution and 0.0001-0.5% (w/w for powder application. Exemplary application of the peptide in solution form is described in Wei, Science 257:85-88 (1992), which is hereby incorporated by reference in its entirety. Briefly, peptides can be dissolved at a concentration of 500 μg/ml in aqueous solution and then introduced onto the leaves of preflowering plants. Leaves can be lightly punctured with a toothpick (i.e., mechanically wounded) in a middle leaf panel, and then peptide solution can be infused via needle-less syringe into the wound, filling the panel. The leaves can be observed and scored over the next 48 hours for withering and browning, lesions typical of programmed cell death. Plants are considered HR-positive (“HR+”) if they exhibit wide-spread macroscopic cell death visible to the naked eye, accompanied by wilting and browning of the affected tissue within 48 hours. Plants are considered HR-negative (“HR−”) if they exhibit no discernible wilting or tissue death observable by naked eye. Weak-HR elicitation is evidenced by minimal browning or withering that is limited in scope after 48 hours.

In certain embodiments, material alteration of the one or more properties is intended to mean that there is less than 20% variation, less than 15% variation, less than 10% variation, or less than 5% variation in a recited property when comparing a peptide possessing the one or more extraneous amino acids to an otherwise identical peptide lacking the one or more extraneous amino acids. In certain embodiments, the number of extraneous amino acids at the N- or C-terminal ends is up to 20 amino acids at one or both ends, up to 15 amino acids at one or both ends, up to 10 amino acids at one or both ends, up to 7 amino acids at one or both ends, up to 5 amino acids at one or both ends, or up to 3 amino acids at one or both ends. Further, to the extent that a recited sequence is in the form of a consensus sequence where one or more of the denoted X or Xaa residues can be any of one or more amino acids, then multiple peptide sequences are embraced by the peptide consisting essentially of such a recited sequence, without regard to additional variations of such sequences that are afforded by the presence of extraneous amino acids at the N- and/or C-terminal ends thereof.

In various embodiments of the invention, the disclosed peptides may include a hydrophilic amino acid sequence, e.g., at either the N-terminal or C-terminal end of a designated peptide sequence. The hydrophilic amino acid sequence is at least 3, at least 4, at least 5, at least 6, at least 7, at least 8, at least 9, or at least 10 amino acids in length, and includes amino acid residues that contribute to a hydrophilic property of the amino acid sequence that is adjacent to the amino acid sequence of the designated peptide (i.e., the peptide that induces an active plant response). Different methods have been used in the art to calculate the relative hydrophobicity/hydrophilicity of amino acid residues and proteins (Kyte et al., “A Simple Method for Displaying the Hydropathic Character of a Protein,” J. Mol. Biol. 157: 105-32 (1982); Eisenberg D, “Three-dimensional Structure of Membrane and Surface Proteins,” Ann. Rev. Biochem. 53: 595-623 (1984); Rose et al., “Hydrogen Bonding, Hydrophobicity, Packing, and Protein Folding,” Annu. Rev. Biomol. Struct. 22: 381-415 (1993); Kauzmann, “Some Factors in the Interpretation of Protein Denaturation,” Adv. Protein Chem. 14: 1-63 (1959), which are hereby incorporated by reference in their entirety). Any one of these hydrophobicity scales can be used for the purposes of the present invention; however, the Kyte-Doolittle hydrophobicity scale is perhaps the most often referenced scale. These hydropathy scales provide a ranking list for the relative hydrophobicity of amino acid residues. For example, amino acids that contribute to hydrophilicity include Arg (R), Lys (K), Asp (D), Glu (E), Gln (Q), Asn (N), and His (H) as well as, albeit to a lesser extent, Ser (S), Thr (T), Gly (G), Pro (P), Tyr (Y), and Trp (W). For example, polyglutamate sequences can be used to enhance solubility of proteins and other drug molecules (Lilie et al, Biological Chemistry 394(8):995-1004(2013); Li et al., Cancer Research 58: 2404-2409(1998)), each of which is hereby incorporated by reference in its entirety).

The “hydropathy index” of a protein or amino acid sequence is a number representing its average hydrophilic or hydrophobic properties. A negative hydropathy index defines the hydrophilicity of the amino acid sequence of interest. The hydropathy index is directly proportional to the hydrophilicity of the amino acid sequence of interest; thus, the more negative the index, the greater its hydrophilicity. In certain embodiments, the added hydrophilic amino acid sequence described above has a hydropathy index of less than 0, −0.4, −0.9, −1.3, −1.6, −3.5, −3.9, or −4.5. In certain embodiments, the resulting entire peptide will have a hydropathy index of less than 0.7, 0.3, 0.2, 0.1, or 0.0, preferably less than −0.1, −0.2, −0.3, −0.4, more preferably less than −0.5, −0.6, −0.7, −0.8, −0.9, or −1.0.

In the peptides of the present invention, amino acids that contribute to a hydrophilic hydropathy index, for either the peptide as a whole or the added hydrophilic amino acid sequence, include Arg (R), Lys (K), Asp (D), Glu (E), Gln (Q), Asn (N), His (H), Ser (S), Thr (T), Gly (G), Pro (P), Tyr (Y), and Trp (W). Of these, Asp (D), Glu (E), Gln (Q), Asn (N) or their variants are preferred. Exemplary variants include g-glutamate for Glu and isoaspartic acid (or isoD) for Asp.

As used herein, in this and in other aspects of the invention, the term “hydrophobic amino acid” is intended to refer to an amino acid that contributes hydrophobicity to the hydropathy index of a designated amino acid sequence. Amino acids that contribute to a hydrophobic hydropathy index, for either the peptide as a whole or a particular amino acid sequence thereof, include Ile (I), Val (V), Leu (L), Phe (F), Cys (C), Met (M), and Ala (A). In certain embodiments, the term “hydrophobic amino acid” may refer to any one of Ile (I), Val (V), Leu (L), Phe (F), Cys (C), Met (M), and Ala (A); or, alternatively, to any one of Ile (I), Val (V), Leu (L), Phe (F), and Ala (A). In certain other embodiments, the term “hydrophobic amino acid” may refer to one of Ile (I), Val (V), Leu (L), and Phe (F).

As used herein, the term “non-hydrophobic amino acid” is intended to mean an amino acid that is hydrophilic (or not hydrophobic) on one of the above-identified hydrophobicity scales. This term generally refers to those amino acids that contribute to a hydrophilic hydropathy index for either the peptide as a whole or the added hydrophilic amino acid sequence.

In one aspect of the invention, the peptide includes the amino acid sequence of (L/M)-X-X-(L/M)-X-X-L-(L/M)-X-(L/I)-(E/L/F)-X-X-(L/I)-X-X-X-L-(L/F) (SEQ ID NO:1), wherein each X is independently any amino acid.

The peptide length in this embodiment is less than 100 amino acids, or alternatively less than 90 amino acids, less than 80 amino acids, less than 70 amino acids, less than 60 amino acids, or less than about 50 amino acids. In certain embodiments, the peptide length is up to 50 amino acids, such as between 19 and about 50 amino acids in length.

In the embodiments described above, where each X of SEQ ID NO: 1 can be any amino acid, in certain embodiments these residues are hydrophilic in nature. As described above, these hydrophilic amino acids include Arg (R), Lys (K), Asp (D), Glu (E), Gln (Q), Asn (N), His (H), Ser (S), Thr (T), Gly (G), Pro (P), Tyr (Y), and Trp (W). Of these, Glu (E), Pro (P), Ser (S), Gln (Q), Lys (K), Asp (D), Thr (T) or their variants are preferred. Exemplary variants include g-glutamate for Glu and isoaspartic acid (or isoD) for Asp. The number of cationic (positively charged) amino acids (generally R or K) should be limited to 2 in order to avoid possible toxicity when applied to plant tissues. Experience with other harpin-derived bioactive peptides, as described in PCT Application Publication Nos. WO2016/054310 and WO2016/054342, which are hereby incorporated by reference in their entirety, has demonstrated that mutation of these residues, particularly to other hydrophilic amino acids (R, K, D, E, Q, N, H, S, T, G, or P) does not generally cause a loss of activity.

In the embodiments described above, where each X of SEQ ID NO: 1 can be any amino acid, in certain embodiments one or more of these residues is hydrophobic in nature. In these embodiments, the hydrophobic residue is preferably Ala (A).

In certain embodiments, X at position 2 is selected from Glu (E) and Ser (S); X at position 3 is selected from Glu (E) and Pro (P); X at position 5 is Glu (E); X at position 6 is selected from Glu (E) and Gln (Q); X at position 9 is selected from Glu (E), Lys (K), and Ala (A); X at position 12 is selected from Glu (E) and Ala (A); X at position 13 is selected from Glu (E) and Asp (D); X at position 15 is selected from Glu (E) and Thr (T); X at position 16 is selected from Glu (E) and Gln (Q); and X at position 17 is selected from Glu (E) and Ser (S). In these embodiments, the residue at position 11 can be E; in alternative embodiments the residue at position 11 is either L or F (and not E); in alternative embodiments the residue at position 11 is either F or E (and not L); and in alternative embodiments the residue at position 11 is F (and not L or E).

In certain embodiments, X at position 2 is selected from Glu (E) and Ser (S); X at position 3 is selected from Glu (E) and Pro (P); X at position 5 is Glu (E); X at position 6 is selected from Glu (E) and Gln (Q); X at position 9 is selected from Glu (E) and Lys (K); X at position 12 is Glu (E); X at position 13 is Glu (E); X at position 15 is Glu (E); X at position 16 is selected from Glu (E) and Gln (Q); and X at position 17 is selected from Glu (E) and Ser (S). In these embodiments, the residue at position 11 can be E; in alternative embodiments the residue at position 11 is either L or F (and not E); in alternative embodiments the residue at position 11 is either F or E (and not L); and in alternative embodiments the residue at position 11 is F (and not L or E).

In certain embodiments, X at position 2 is selected from Glu (E) and Ser (S); X at position 3 is selected from Glu (E) and Pro (P); X at position 5 is Glu (E); X at position 6 is selected from Glu (E) and Gln (Q); X at position 9 is selected from Glu (E) and Lys (K); X at position 12 is selected from Ala (A) and Glu (E); X at position 13 is selected from Asp (D) and Glu (E); X at position 15 is selected from Thr (T) and Glu (E); X at position 16 is selected from Glu (E) and Gln (Q); and X at position 17 is selected from Glu (E) and Ser (S). In these embodiments, the residue at position 11 can be E; in alternative embodiments the residue at position 11 is either L or F (and not E); in alternative embodiments the residue at position 11 is either F or E (and not L); and in alternative embodiments the residue at position 11 is F (and not L or E).

One set of peptides according to the first aspect of the invention have the amino acid sequence of: (L/M)-X-X-(L/M)-E-(E/Q)-L-(L/M)-X-(L/I)-(E/L/F)-X-X-(L/I)-X-(E/Q)-X-L-(L/F) (SEQ ID NO: 2) wherein each X is independently any amino acid.

In the embodiments described above, where each X of SEQ ID NO: 2 can be any amino acid, in certain embodiments these residues are hydrophilic in nature. As described above, these hydrophilic amino acids include Arg (R), Lys (K), Asp (D), Glu (E), Gln (Q), Asn (N), His (H), Ser (S), Thr (T), Gly (G), Pro (P), Tyr (Y), and Trp (W). Of these, Glu (E), Pro (P), Ser (S), Lys (K), Asp (D), Thr (T) or their variants are preferred. Exemplary variants include g-glutamate for Glu and isoaspartic acid (or isoD) for Asp. The number of cationic (positively charged) amino acids (generally R or K) should be limited to 2 in order to avoid possible toxicity when applied to plant tissues.

In the embodiments described above, where each X of SEQ ID NO: 2 can be any amino acid, in certain embodiments one or more of these residues is hydrophobic in nature. In these embodiments, the hydrophobic residue is preferably Ala (A).

In certain embodiments, X at position 2 is selected from Glu (E) and Ser (S); X at position 3 is selected from Glu (E) and Pro (P); X at position 9 is selected from Glu (E), Lys (K), and Ala (A); X at position 12 is selected from Glu (E) and Ala (A); X at position 13 is selected from Glu (E) and Asp (D); X at position 15 is selected from Glu (E) and Thr (T); and X at position 17 is selected from Glu (E) and Ser (S). In these embodiments, the residue at position 11 can be E; in alternative embodiments the residue at position 11 is either L or F (and not E); in alternative embodiments the residue at position 11 is either F or E (and not L); and in alternative embodiments the residue at position 11 is F (and not L or E).

In this embodiment, the isolated peptide is stable when dissolved in water; resistant to chemical degradation in aqueous conditions in the presence of a pH buffer and a biocide, as described above; and/or has a solubility in an aqueous solution of at least about 1.0 mg/ml.

In certain embodiments, the peptides according to SEQ ID NOS: 1 or 2 include from 1 to 20 (such as 1 to 15) additional amino acids at the N-terminal end, from 1 to 20 (such as 1 to 15) amino acids at the C-terminal end, or both 1 to 20 (such as 1 to 15) additional amino acids at the N-terminal end and 1 to 20 (such as 1 to 15) amino acids at the C-terminal end. By way of example, the peptides according to SEQ ID NOS: 1 or 2 may include from 1 to 10 additional amino acids at the N-terminal end (such as 1, 2, 3, 4, 5, 6, 7, 8, 9, or 10 amino acids), from 1 to 10 additional amino acids at the C-terminal end (such as 1, 2, 3, 4, 5, 6, 7, 8, 9, or 10 amino acids), or both 1 to 10 additional amino acids at the N-terminal end and 1 to 10 additional amino acids at the C-terminal end as described above. Such peptides therefore vary in length from 20 amino acids up to 59 amino acids, preferably up to 50 amino acids. In these various embodiments, the additional amino acids are preferably hydrophilic amino acids as described above, and more preferably Glu (E), Pro (P), Gly (G), Ser (S), Gln (Q), Lys (K), Asp (D), Thr (T), g-glutamate, or isoaspartic acid (isoD). In certain embodiments, the peptide includes no internal Lys (K) or Arg (R) residues.

In certain embodiments, there are 6 or more additional amino acids at the N-terminal end and 3 or more additional amino acids at the C-terminal end. The additional amino acids are preferably hydrophilic amino acids, as described above. The 6 or more amino acids at the N-terminal end preferably includes the amino acid sequence of (S/A/E/G)-(G/S/E)-(E/Q)-(T/E)-(S/E)-(G/E) (SEQ ID NO: 84). The 3 or more additional amino acids at the C-terminal end preferably includes (G/E)-(D/E)-(Q/E)-(D/E)-(G/E) (SEQ ID NO: 85). In certain embodiments, the last two amino acid residues at the C-terminal end are optional.

In certain embodiments, there are 3 or more additional amino acids at the N-terminal end and 1 or more additional amino acids at the C-terminal end. The additional amino acids are preferably hydrophilic amino acids, as described above. The 3 or more amino acids at the N-terminal end preferably includes the amino acid sequence of (T/E)-(S/E)-(G/E). The 1 or more additional amino acids at the C-terminal end preferably includes (G/E). In certain embodiments, the additional amino acid residues at the N-terminal are optional.

Exemplary peptides that meet the consensus structure of SEQ ID NO: 1 or 2 are identified in Table 1 below:

TABLE 1
Peptide Variants of Peptide P12/P13 (SEQ ID NOs: 1 and 2)

| Peptide Name | Sequence | SEQ ID NO: |
|---|---|---|
| wildtype | QTGDDSLSGAGQTSGMSPMEQLMKIFADITQSLFGDQDG | 5 |
| P12 | QTGDDSLSGAGQTSGMSPMEQLMKIFADITQSLFGDQDG | 5 |
| P12-2 | GDLQGSGASTQDTSGMSPMEQLMKIFADITQSLFGDQDG | 6 |
| P13 | TSGMSPMEQLMKIFADITQSLFG | 7 |
| P13-2 | TSGLSPLEQLLKIFADITQSLFG | 8 |
| P13-3 | TSGLSPLEQLLKIFAEITQSLFG | 9 |
| P13-4 | MSPMEQLMKIFADITQSLFEEEE | 10 |
| P13-5 | LSPLEQLMKIFADITQSLFEEEE | 11 |
| P13-6 | MEEMEELMEIFEEIEEELFEE | 12 |
| P13-7 | LEELEELLEIFEEIEEELFEE | 13 |
| P13-8 | SEEEEMSPMEQLMKIFADITQSLF | 14 |
| P13-9 | SEEEEMSPMEQLMKIFAEITQSLF | 15 |
| P13-10 | DDSLSGAGQTSGMSPMEQLMKIFADITQSLFGDQDG | 16 |
| P13-11 | LSGAGQTSGMSPMEQLMKIFADITQSLFGDQDG | 17 |
| P13-12 | AGQTSGMSPMEQLMKIFADITQSLFGDQDG | 18 |
| P13-13 | QTGDDSLSGAGQTSGMSPMEQLMKIFADITQSLFGDQ | 19 |
| P13-14 | QTGDDSLSGAGQTSGMSPMEQLMKIFADITQSLFG | 20 |
| P13-15 | GQTSGMSPMEQLMKIFADITQSLFG | 21 |
| P13-16 | AGQTSGMSPMEQLMKIFADITQSLFG | 22 |
| P13-17 | AGQTSGMSPMEQLMKIFADITQSLFGDQ | 23 |
| P13-18 | AGQTSGMSPMEQLMEIFADITQSLFGDQDG | 24 |
| P13-19 | AGQTSGMSPMEQLMAIFADITQSLFGDQDG | 25 |
| P13-20 | AGQTSGMSPMEQLMEIFADITQSLFGDQDGR | 26 |
| P13-21 | AGQTSGMSPMEQLMAIFADITQSLFGDQDGR | 27 |
| P13-22 | AGQTSGMSPMEQLMEIFADITQSLFGDQDGK | 28 |
| P13-23 | AGQTSGLSPLEQLLKIFADITQSLFG | 29 |
| P13-24 | GQTSGMSPMEQLMEIFADITQSLF | 30 |
| P13-25 | SQTSGMSPMEQLMEIFADITQSLF | 31 |
| P13-26 | SQEEEMEPMEQLMEIFEEIEQELFG | 32 |
| P13-27 | SQEEEMEEMEQLMEIFEEIEQELFG | 33 |
| P13-28 | SEQEEEMEEMEQLMEIFEEIEQELFE | 34 |
| P13-29 | SEQEEELEELEQLLEIFEEIEQELFE | 35 |
| P13-30 | AGQTSGMSPMEQLMKLFADLTQSLFGDQDG | 36 |
| P13-31 | AGQTSGMSPMEQLMKILADITQSLFGDQDG | 37 |
| P13-32 | AGQTSGMSPMEQLMKIFADITQSLLGDQDG | 38 |
| P13-33 | AGQTSGMSPMEQLMKILADITQSLLGDQDG | 39 |
| P13-34 | SEQEEEMEEMEQLMEIFEEIEQELF | 40 |
| P13-35 | LEELEELLEIFEEIEEELF | 41 |
| P13-36 | SEEMSPMEQLMKIFADITQSLFEE | 42 |
| P13-37 | MEEMEQLMKIFEEIEQELFEEEE | 43 |
| P13-38 | MSPMEELMKIFADITESLFEEEE | 44 |
| P13-s5 | MSPMEQLMKIFADITQSLFEE | 45 |
| P13-s6 | MEEMEQLMEIFEEIEQELFEEEE | 46 |
| P13-s7 | MSPMEQLMEIFADITQSLFEEEE | 47 |
| P13-s8 | LEEMEELMEIFEEIEEELFEE | 48 |
| P13-s9 | MEELEELMEIFEEIEEELFEE | 49 |
| P13-s10 | MEEMEELLEIFEEIEEELFEE | 50 |
| P13-s11 | MEELEELLEIFEEIEEELFEE | 51 |
| P13-s12 | LEEMEELLEIFEEIEEELFEE | 52 |
| P13-s13 | LEELEELMEIFEEIEEELFEE | 53 |
| P13-s14 | TSGLSPLEQLLEIFADITQSLFGR | 83 |
| P13-s15 | TSGLSPLEQLLEIFAEITQSLFGR | 84 |
| P13-39 | AGQTSGMSPMEQLLKIFADITQSLFGDQDG | 61 |
| P13-40 | AGQTSGMSPLEQLMKIFADITQSLFGDQDG | 62 |
| P13-41 | AGQTSGLSPMEQLMKIFADITQSLFGDQDG | 63 |
| P13-42 | AGETSGMSPMEQLMKIFADITQSLFGDQDG | 64 |
| P13-43 | AGQTSGMSPMEQLMKIFADITESLFGDQDG | 65 |
| P13-44 | AGQTSGMSPMEQLMKIFADITQSLFGDEDG | 66 |
| P13-45 | AGQTSGMSPMEELMKIFADITQSLFGDQDG | 67 |
| P13-46 | AEQEEEMEPMEQLMKIFEEIEQELFEEEEE | 68 |
| P13-52 | AGQTSGMSPMEQLMKIEADITQSLFGDQDG | 74 |
| P13-56 | AGQTSGMSPMEQLMEIFADITQSLFGDQDR | 78 |
| P13-57 | AGQTSGMSPMEQLMEIFADITQSLFGDQR | 79 |
| P13-58 | AGQTSGMSPMEQLMEIFADITQSLFGDR | 80 |

Select peptides in Table 1 include solubility tags, indicated by italic print, including SE, SEE, and SEEEE (SEQ ID NO: 81), as well as EE and EEEE (SEQ ID NO: 82); or cleavage tags, indicated by italic print, including a C-terminal R or K. Peptides comprising the sequences shown in Table 1 but lacking these specific solubility or cleavage tags (or having a different tag) are also contemplated herein.

Another set of peptides according to the first aspect of the invention have the amino acid sequence of: (L/M)-X-X-(L/M)-E-X-L-(L/M)-X-I-F-X-X-I-X-X-X-L-F (SEQ ID NO:3), wherein each X is independently one of R, K, D, E, Q, N, H, S, T, G, P, Y, W, or A. The number of cationic (positively charged) amino acids (generally R or K) should be limited to 2 in order to avoid possible toxicity when applied to plant tissues.

In certain embodiments, X at position 2 is selected from Glu (E) and Ser (S); X at position 3 is selected from Glu (E) and Pro (P); X at position 6 is selected from Glu (E) and Gln (Q); X at position 9 is selected from Glu (E) and Lys (K); X at position 12 is selected from Glu (E) and Ala (A); X at position 13 is selected from Glu (E) and Asp (D); X at position 15 is selected from Glu (E) and Thr (T); X at position 16 is selected from Glu (E) and Gln (Q); and X at position 17 is selected from Glu (E) and Ser (S).

In certain embodiments, X at position 2 is selected from Glu (E) and Ser (S); X at position 3 is selected from Glu (E) and Pro (P); X at position 6 is selected from Glu (E) and Gln (Q); X at position 9 is selected from Glu (E) and Lys (K); X at position 12 is Glu (E); X at position 13 is Glu (E); X at position 15 is Glu (E); X at position 16 is selected from Glu (E) and Gln (Q); and X at position 17 is selected from Glu (E) and Ser (S).

In certain embodiments, X at position 2 is selected from Glu (E) and Ser (S); X at position 3 is selected from Glu (E) and Pro (P); X at position 6 is selected from Glu (E) and Gln (Q); X at position 9 is selected from Glu (E) and Lys (K); X at position 12 is selected from Ala (A) and Glu (E); X at position 13 is selected from Asp (D) and Glu (E); X at position 15 is selected from Thr (T) and Glu (E); X at position 16 is selected from Glu (E) and Gln (Q); and X at position 17 is selected from Glu (E) and Ser (S).

In certain embodiments, the peptides according to SEQ ID NO: 3 include from 1 to 20 (such as 1 to 15) additional amino acids at the N-terminal end, from 1 to 20 (such as 1 to 15) amino acids at the C-terminal end, or both 1 to 20 (such as 1 to 15) additional amino acids at the N-terminal end and 1 to 20 (such as 1 to 15) amino acids at the C-terminal end. By way of example, the peptides according to SEQ ID NO: 3 may include from 1 to 10 additional amino acids at the N-terminal end (such as 1, 2, 3, 4, 5, 6, 7, 8, 9, or 10 amino acids), from 1 to 10 additional amino acids at the C-terminal end (such as 1, 2, 3, 4, 5, 6, 7, 8, 9, or 10 amino acids), or both 1 to 10 additional amino acids at the N-terminal end and 1 to 10 additional amino acids at the C-terminal end as described above. Such peptides therefore vary in length from 20 amino acids up to 59 amino acids, preferably up to 50 amino acids. In these various embodiments, the additional amino acids are preferably hydrophilic amino acids as described above, and more preferably Glu (E), Pro (P), Gly (G), Ser (S), Gln (Q), Lys (K), Asp (D), Thr (T), g-glutamate, or isoaspartic acid (isoD). In certain embodiments, the peptide includes no internal Lys (K) or Arg (R) residues.

In certain embodiments, there are 6 or more additional amino acids at the N-terminal end and 3 or more additional amino acids at the C-terminal end. The additional amino acids are preferably hydrophilic amino acids, as described above. The 6 or more amino acids at the N-terminal end preferably includes the amino acid sequence of (S/A/E/G)-(G/S/E)-(E/Q)-(T/E)-(S/E)-(G/E) (SEQ ID NO: 84). The 3 or more additional amino acids at the C-terminal end preferably includes (G/E)-(D/E)-(Q/E)-(D/E)-(G/E) (SEQ ID NO: 85). In certain embodiments, the last two amino acid residues at the C-terminal end are optional.

In certain embodiments, there are 3 or more additional amino acids at the N-terminal end and 1 or more additional amino acids at the C-terminal end. The additional amino acids are preferably hydrophilic amino acids, as described above. The 3 or more amino acids at the N-terminal end preferably includes the amino acid sequence of (T/E)-(S/E)-(G/E). The 1 or more additional amino acids at the C-terminal end preferably includes (G/E). In certain embodiments, the additional amino acid residues at the N-terminal are optional.

Exemplary peptides that meet the consensus structure of SEQ ID NO: 3 are identified in Table 2 below:

TABLE 2
Peptide Variants of Peptide P12/P13 (SEQ ID NO: 3)

| Peptide Name | Sequence | SEQ ID NO: |
|---|---|---|
| wildtype | QTGDDSLSGAGQTSGMSPMEQLMKIFADITQSLFGDQDG | 5 |
| P12 | QTGDDSLSGAGQTSGMSPMEQLMKIFADITQSLFGDQDG | 5 |
| P12-2 | GDLQGSGASTQDTSGMSPMEQLMKIFADITQSLFGDQDG | 6 |
| P13 | TSGMSPMEQLMKIFADITQSLFG | 7 |
| P13-2 | TSGLSPLEQLLKIFADITQSLFG | 8 |
| P13-3 | TSGLSPLEQLLKIFAEITQSLFG | 9 |
| P13-4 | MSPMEQLMKIFADITQSLFEEEE | 10 |
| P13-5 | LSPLEQLMKIFADITQSLFEEEE | 11 |
| P13-6 | MEEMEELMEIFEEIEEELFEE | 12 |
| P13-7 | LEELEELLEIFEEIEEELFEE | 13 |
| P13-8 | SEEEEMSPMEQLMKIFADITQSLF | 14 |
| P13-9 | SEEEEMSPMEQLMKIFAEITQSLF | 15 |
| P13-10 | DDSLSGAGQTSGMSPMEQLMKIFADITQSLFGDQDG | 16 |
| P13-11 | LSGAGQTSGMSPMEQLMKIFADITQSLFGDQDG | 17 |
| P13-12 | AGQTSGMSPMEQLMKIFADITQSLFGDQDG | 18 |
| P13-13 | QTGDDSLSGAGQTSGMSPMEQLMKIFADITQSLFGDQ | 19 |
| P13-14 | QTGDDSLSGAGQTSGMSPMEQLMKIFADITQSLFG | 20 |
| P13-15 | GQTSGMSPMEQLMKIFADITQSLFG | 21 |
| P13-16 | AGQTSGMSPMEQLMKIFADITQSLFG | 22 |
| P13-17 | AGQTSGMSPMEQLMKIFADITQSLFGDQ | 23 |
| P13-18 | AGQTSGMSPMEQLMEIFADITQSLFGDQDG | 24 |
| P13-19 | AGQTSGMSPMEQLMAIFADITQSLFGDQDG | 25 |
| P13-20 | AGQTSGMSPMEQLMEIFADITQSLFGDQDGR | 26 |
| P13-21 | AGQTSGMSPMEQLMAIFADITQSLFGDQDGR | 27 |
| P13-22 | AGQTSGMSPMEQLMEIFADITQSLFGDQDGK | 28 |
| P13-23 | AGQTSGLSPLEQLLKIFADITQSLFG | 29 |
| P13-24 | GQTSGMSPMEQLMEIFADITQSLF | 30 |
| P13-25 | SQTSGMSPMEQLMEIFADITQSLF | 31 |
| P13-26 | SQEEEMEPMEQLMEIFEEIEQELFG | 32 |
| P13-27 | SQEEEMEEMEQLMEIFEEIEQELFG | 33 |
| P13-28 | SEQEEEMEEMEQLMEIFEEIEQELFE | 34 |
| P13-29 | SEQEEELEELEQLLEIFEEIEQELFE | 35 |
| P13-34 | SEQEEEMEEMEQLMEIFEEIEQELF | 40 |
| P13-35 | LEELEELLEIFEEIEEELF | 41 |
| P13-36 | SEEMSPMEQLMKIFADITQSLFEE | 42 |
| P13-37 | MEEMEQLMKIFEEIEQELFEEEE | 43 |
| P13-38 | MSPMEELMKIFADITESLFEEEE | 44 |
| P13-s5 | MSPMEQLMKIFADITQSLFEE | 45 |
| P13-s6 | MEEMEQLMEIFEEIEQELFEEEE | 46 |
| P13-s7 | MSPMEQLMEIFADITQSLFEEEE | 47 |
| P13-s8 | LEEMEELMEIFEEIEEELFEE | 48 |
| P13-s9 | MEELEELMEIFEEIEEELFEE | 49 |
| P13-s10 | MEEMEELLEIFEEIEEELFEE | 50 |
| P13-s11 | MEELEELLEIFEEIEEELFEE | 51 |
| P13-s12 | LEEMEELLEIFEEIEEELFEE | 52 |
| P13-s13 | LEELEELMEIFEEIEEELFEE | 53 |
| P13-s14 | TSGLSPLEQLLEIFADITQSLFGR | 83 |
| P13-s15 | TSGLSPLEQLLEIFAEITQSLFGR | 84 |
| P13-39 | AGQTSGMSPMEQLLKIFADITQSLFGDQDG | 61 |
| P13-40 | AGQTSGMSPLEQLMKIFADITQSLFGDQDG | 62 |
| P13-41 | AGQTSGLSPMEQLMKIFADITQSLFGDQDG | 63 |
| P13-42 | AGETSGMSPMEQLMKIFADITQSLFGDQDG | 64 |
| P13-43 | AGQTSGMSPMEQLMKIFADITESLFGDQDG | 65 |
| P13-44 | AGQTSGMSPMEQLMKIFADITQSLFGDEDG | 66 |
| P13-45 | AGQTSGMSPMEELMKIFADITQSLFGDQDG | 67 |
| P13-46 | AEQEEEMEPMEQLMKIFEEIEQELFEEEEE | 68 |
| P13-56 | AGQTSGMSPMEQLMEIFADITQSLFGDQDR | 78 |
| P13-57 | AGQTSGMSPMEQLMEIFADITQSLFGDQR | 79 |
| P13-58 | AGQTSGMSPMEQLMEIFADITQSLFGDR | 80 |

Select peptides in Table 1 include solubility tags, indicated by italic print, including SE, SEE, and SEEEE (SEQ ID NO: 81), as well as EE and EEEE (SEQ ID NO: 82); or cleavage tags, indicated by italic print, including a C-terminal R or K. Peptides comprising the sequences shown in Table 1 but lacking these specific solubility or cleavage tags (or having a different tag) are also contemplated herein.

A further set of peptides according to the first aspect of the invention have the amino acid sequence of: T-S-G-(L/M)-S-P-(L/M)-E-Q-L-(L/M)-K-I-F-A-D-I-T-Q-S-L-F (SEQ ID NO: 4).

Exemplary peptides that meet the consensus structure of SEQ ID NO: 4 are identified in Table 3 below:

TABLE 3
Peptide Variants of Peptide P12/P13 (SEQ ID NO: 4)

| Peptide | | SEQ ID |
|---|---|---|
| Name | Sequence | NO: |
| wildtype | QTGDDSLSGAGQTSGMSPMEQLMKIFADITQSLFGDQDG | 5 |
| P12 | QTGDDSLSGAGQTSGMSPMEQLMKIFADITQSLFGDQDG | 5 |
| P12-2 | GDLQGSGASTQDTSGMSPMEQLMKIFADITQSLFGDQDG | 6 |
| P13 | TSGMSPMEQLMKIFADITQSLFG | 7 |
| P13-2 | TSGLSPLEQLLKIFADITQSLFG | 8 |
| P13-10 | DDSLSGAGQTSGMSPMEQLMKIFADITQSLFGDQDG | 16 |
| P13-11 | LSGAGQTSGMSPMEQLMKIFADITQSLFGDQDG | 17 |
| P13-12 | AGQTSGMSPMEQLMKIFADITQSLFGDQDG | 18 |
| P13-13 | QTGDDSLSGAGQTSGMSPMEQLMKIFADITQSLFGDQ | 19 |
| P13-14 | QTGDDSLSGAGQTSGMSPMEQLMKIFADITQSLFG | 20 |
| P13-15 | GQTSGMSPMEQLMKIFADITQSLFG | 21 |
| P13-16 | AGQTSGMSPMEQLMKIFADITQSLFG | 22 |
| P13-17 | AGQTSGMSPMEQLMKIFADITQSLFGDQ | 23 |
| P13-23 | AGQTSGLSPLEQLLKIFADITQSLFG | 29 |
| P13-39 | AGQTSGMSPMEQLLKIFADITQSLFGDQDG | 61 |
| P13-40 | AGQTSGMSPLEQLMKIFADITQSLFGDQDG | 62 |
| P13-41 | AGQTSGLSPMEQLMKIFADITQSLFGDQDG | 63 |
| P13-42 | AGETSGMSPMEQLMKIFADITQSLFGDQDG | 64 |
| P13-44 | AGQTSGMSPMEQLMKIFADITQSLFGDEDG | 66 |

In certain embodiments, the peptide includes one or more mutations relative to the corresponding wildtype amino acid sequence of:

(SEQ ID NO: 5)
QTGDDSLSGAGQTSGMSPMEQLMKIFADITQSLFGDQDG,

which corresponds to amino acid residues 123-161 of the HrpN protein of Pantoea stewartii (formerly a member of genus Erwinia, sequence detailed in Frederick et al, Mol Plant Microbe Interact. 14(10):1213-22 (2001), which is hereby incorporated by reference in its entirety). These one or more mutations include, in addition to truncation of the full length 382 aa HrpN protein at one or both of its N-terminal and C-terminal ends, one or more deletions or substitutions relative to SEQ ID NO: 5. In certain embodiments, the one or more mutations improve the solubility in aqueous solution, stability, and/or resistance to chemical degradation of the isolated peptide relative to a polypeptide comprising or consisting of the corresponding wildtype amino acid sequence of SEQ ID NO: 5. In this embodiment, the isolated peptide is stable when dissolved in water; resistant to chemical degradation in aqueous conditions in the presence of a pH buffer and a biocide, as described above; and/or has a solubility in an aqueous solution of at least about 1.0 mg/ml.

The isolated peptides of the invention can also be presented in the form of a fusion peptide that includes, in addition, a second amino acid sequence coupled to the inventive peptides via peptide bond. The second amino acid sequence can be a purification tag, such as poly-histidine (His₆-), a glutathione-S-transferase (GST-), or maltose-binding protein (MBP-), which assists in the purification but can later be removed, i.e., cleaved from the peptide following recovery. Protease-specific cleavage sites or chemical-specific cleavage sites (i.e., in a cleavable linker sequence) can be introduced between the purification tag and the desired peptide. Protease-specific cleavage sites are well known in the literature and include, without limitation, the enterokinase specific cleavage site (Asp)₄-Lys (SEQ ID NO: 54), which is cleaved after lysine; the factor Xa specific cleavage site Ile-(Glu or Asp)-Gly-Arg (SEQ ID NO: 55), which is cleaved after arginine; the trypsin specific cleavage site, which cleaves after Lys and Arg; and the GenenaseTM I specific cleavage site Pro-Gly-Ala-Ala-His-Tyr (SEQ ID NO: 56). Chemicals and their specific cleavage sites include, without limitation, cyanogen bromide (CNBr), which cleaves at methionine (Met) residues; BNPS-skatole, which cleaves at tryptophan (Trp) residues; formic acid, which cleaves at aspartic acid-proline (Asp-Pro) peptide bonds; hydroxylamine, which cleaves at asparagine-glycine (Asn-Gly) peptide bonds; and 2-nitro-5-thiocyanobenzoic acid (NTCB), which cleaves at cysteine (Cys) residues (see Crimmins et al., “Chemical Cleavage of Proteins in Solution,” Curr. Protocol. Protein Sci., Chapter 11:Unit 11.4 (2005), which is hereby incorporated by reference in its entirety). In order to use one of these cleavage methods, it may be necessary to remove unwanted cleavage sites from within the desired peptide sequences by mutation. For example, the peptide sequence may comprise an arginine or lysine residue at the C-terminal end and also have any lysine or arginine residues changed to E, D, S, T, A, G, N, Q (preferably) or any other amino acid that eliminates unwanted trypsin cleavage sites from within the peptide sequence. Thus, P13-18 (SEQ ID NO: 24) and P13-19 (SEQ ID NO: 25) are mutant sequences derived from P12 with the lysine residue mutated to either glutamic acid or alanine. Peptides comprising this sequence can be produced by trypsin-mediated cleavage of a tandem repeated sequence of P13-18 separated by lysine or arginine residues. The residual peptide following trypsin-mediated cleavage will contain a lysine or arginine residue at the site of such cleavage, which is illustrated, for example, by P13-20 (SEQ ID NO: 26), P13-21 (SEQ ID NO: 27), and P13-22 (SEQ ID NO: 28). When designing peptides for cleavage with trypsin, care should be taken regarding solubility tags incorporating negatively charged residues near the cleavage sites. Ion pairing between the cleavage site R or K with a negatively-charged amino acid has been shown to reduce the efficiency of trypsin cleavage as described by S̆lechtova et al., Analytical Chemistry 87:7636-43 (2015), which is hereby incorporated by reference in its entirety.

The isolated peptides of the invention can also be presented in the form of a fusion peptide that includes multiple peptide sequences of the present invention linked together by a linker sequence, which may or may not take the form of a cleavable amino acid sequence of the type described above. Such multimeric fusion polypeptides may or may not include purification tags. In one embodiment, each monomeric sequence can include a purification tag linked to a peptide of the invention by a first cleavable peptide sequence; and the several monomeric sequences can be linked to adjacent monomeric sequences by a second cleavable peptide sequence. Consequently, upon expression of the multimeric fusion polypeptide, i.e., in a host cell, the recovered fusion polypeptide can be treated with a protease or chemical that is effective to cleave the second cleavable peptide sequence, thereby releasing individual monomeric peptide sequences containing purification tags. Upon affinity purification, the recovered monomeric peptide sequences can be treated with a protease or chemical that is effective to cleave the first cleavable peptide sequence and thereby release the purification tag from the peptide of interest. The latter can be further purified using gel filtration and/or HPLC as described infra.

According to one approach, the peptides of the present invention can be synthesized by standard peptide synthesis operations. These include both FMOC (9-fluorenylmethyloxy-carbonyl) and tBoc (tert-butyloxy-carbonyl) synthesis protocols that can be carried out on automated solid phase peptide synthesis instruments including, without limitation, the Applied Biosystems 431A, 433A synthesizers and Peptide Technologies Symphony or large scale Sonata or CEM Liberty automated solid phase peptide synthesizers. The use of alternative peptide synthesis instruments is also contemplated. Peptides prepared using solid phase synthesis are recovered in a substantially pure form.

The peptides of the present invention may be also prepared by using recombinant expression systems followed by separation and purification of the recombinantly prepared peptides. Generally, this involves inserting an encoding nucleic acid molecule into an expression system to which the molecule is heterologous (i.e., not normally present). One or more desired nucleic acid molecules encoding a peptide of the invention may be inserted into the vector. The heterologous nucleic acid molecule is inserted into the expression system or vector in proper sense (5′-3′) orientation and correct reading frame relative to the promoter and any other 5′ and 3′ regulatory molecules.

Representative nucleotide sequences for expression in representative bacteria and plant hosts are included in Table 4 below:

TABLE 4

| Peptide & | | |
|---|---|---|
| Optimized Host | Nucleotide Sequence | SEQ ID NO: |
| P12 in E. coli | CAGACCGGTGATGATAGCCTGAGCGGTGCAGGTCAGA | 57 |
| | CCAGCGGTATGAGCCCGATGGAACAGCTGATGAAAAT | |
| | TTTTGCAGATATTACCCAGAGCCTGTTTGGTGATCAG | |
| | GATGGT | |
| P13-20 in E. coli | GCAGGTCAGACCAGCGGTATGAGCCCGATGGAACAGC | 58 |
| | TGATGGAAATTTTTGCAGATATTACCCAGAGCCTGTT | |
| | TGGTGATCAGGATGGTCGT | |
| P12 in Zea mays | CAGACCGGCGACGACTCCCTGTCCGGCGCCGGCCAGA | 59 |
| | CCTCCGGCATGTCCCCGATGGAGCAGCTGATGAAGAT | |
| | CTTCGCCGACATCACCCAGTCCCTGTTCGGCGACCAG | |
| | GACGGC | |
| P13-20 in Zea mays | GCCGGCCAGACCTCCGGCATGTCCCCGATGGAGCAGC | 60 |
| | TGATGGAGATCTTCGCCGACATCACCCAGTCCCTGTT | |
| | CGGCGACCAGGACGGCAGG | |

With knowledge of the encoded amino acid sequence listed herein and the desired transgenic organism, additional codon-optimized DNA sequences and RNA sequences can be generated with nothing more than routine skill.

Expression (including transcription and translation) of a peptide or fusion polypeptide of the invention by the DNA construct may be regulated with respect to the level of expression, the tissue type(s) where expression takes place and/or developmental stage of expression. A number of heterologous regulatory sequences (e.g., promoters and enhancers) are available for controlling the expression of the DNA construct in plants. These include constitutive, inducible and regulatable promoters, as well as promoters and enhancers that control expression in a tissue- or temporal-specific manner. Exemplary constitutive promoters include the raspberry E4 promoter (U.S. Pat. Nos. 5,783,393 and 5,783,394, each of which is hereby incorporated by reference in its entirety), the nopaline synthase (NOS) promoter (Ebert et al., Proc. Natl. Acad. Sci. (U.S.A.) 84:5745-5749 (1987), which is hereby incorporated by reference in its entirety), the octopine synthase (OCS) promoter (which is carried on tumor-inducing plasmids of Agrobacterium tumefaciens), the caulimovirus promoters such as the cauliflower mosaic virus (CaMV) 19S promoter (Lawton et al., Plant Mol. Biol. 9:315-324 (1987), which is hereby incorporated by reference in its entirety) and the CaMV 35S promoter (Odell et al., Nature 313:810-812 (1985), which is hereby incorporated by reference in its entirety), the figwort mosaic virus 35S-promoter (U.S. Pat. No. 5,378,619, which is hereby incorporated by reference in its entirety), the light-inducible promoter from the small subunit of ribulose-1,5-bis-phosphate carboxylase (ssRUBISCO), the Adh promoter (Walker et al., Proc. Natl. Acad. Sci. (U.S.A.) 84:6624-6628 (1987), which is hereby incorporated by reference in its entirety), the sucrose synthase promoter (Yang et al., Proc. Natl. Acad. Sci. (U.S.A.) 87:4144-4148 (1990), which is hereby incorporated by reference in its entirety), the R gene complex promoter (Chandler et al., Plant Cell 1:1175-1183 (1989), which is hereby incorporated by reference in its entirety), the chlorophyll a/b binding protein gene promoter, the CsVMV promoter (Verdaguer et al., Plant Mol Biol., 37:1055-1067 (1998), which is hereby incorporated by reference in its entirety), and the melon actin promoter (PCT Publ. No. WO00/56863, which is hereby incorporated by reference in its entirety). Exemplary tissue-specific promoters include the tomato E4 and E8 promoters (U.S. Pat. No. 5,859,330, which is hereby incorporated by reference in its entirety) and the tomato 2AII gene promoter (Van Haaren et al., Plant Mol Bio., 21:625-640 (1993), which is hereby incorporated by reference in its entirety).

In one preferred embodiment, expression of the DNA construct is under control of regulatory sequences from genes whose expression is associated with early seed and/or embryo development. Indeed, in a preferred embodiment, the promoter used is a seed-enhanced promoter. Examples of such promoters include the 5′ regulatory regions from such genes as napin (Kridl et al., Seed Sci. Res. 1:209:219 (1991), which is hereby incorporated by reference in its entirety), globulin (Belanger and Kriz, Genet. 129: 863-872 (1991), GenBank Accession No. L22295, each of which is hereby incorporated by reference in its entirety), gamma zein Z 27 (Lopes et al., Mol Gen Genet. 247:603-613 (1995), which is hereby incorporated by reference in its entirety), L3 oleosin promoter (U.S. Pat. No. 6,433,252, which is hereby incorporated by reference in its entirety), phaseolin (Bustos et al., Plant Cell 1(9):839-853 (1989), which is hereby incorporated by reference in its entirety), arcelin5 (U.S. Application Publ. No. 2003/0046727, which is hereby incorporated by reference in its entirety), a soybean 7S promoter, a 7Sa promoter (U.S. Application Publ. No. 2003/0093828, which is hereby incorporated by reference in its entirety), the soybean 7Sαβ conglycinin promoter, a 7Sα a promoter (Beachy et al., EMBO J. 1 4:3047 (1985); Schuler et al., Nucleic Acid Res. 10(24):8225-8244 (1982), each of which is hereby incorporated by reference in its entirety), soybean trypsin inhibitor (Riggs et al., Plant Cell 1(6):609-621 (1989), which is hereby incorporated by reference in its entirety), ACP (Baerson et al., Plant Mol. Biol., 22(2):255-267 (1993), which is hereby incorporated by reference in its entirety), stearoyl-ACP desaturase (Slocombe et al., Plant Physiol. 104(4):167-176 (1994), which is hereby incorporated by reference in its entirety), soybean a′ subunit of β-conglycinin (Chen et al., Proc. Natl. Acad. Sci. 83:8560-8564 (1986), which is hereby incorporated by reference in its entirety), Vicia faba USP (U.S. Application Publ. No. 2003/229918, which is hereby incorporated by reference in its entirety) and Zea mays L3 oleosin promoter (Hong et al., Plant Mol. Biol., 34(3):549-555 (1997), which is hereby incorporated by reference in its entirety).

Nucleic acid molecules encoding the peptides of the present invention can be prepared via solid-phase synthesis using, e.g., the phosphoramidite method and phosphoramidite building blocks derived from protected 2′-deoxynucleosides. To obtain the desired oligonucleotide, the building blocks are sequentially coupled to the growing oligonucleotide chain in the order required by the sequence of the product. Upon the completion of the chain assembly, the product is released from the solid phase to solution, deprotected, collected, and typically purified using HPLC. The limits of solid phase synthesis are suitable for preparing oligonucleotides up to about 200 nt in length, which encodes peptides on the order of about 65 amino acids or less. The ends of the synthetized oligonucleotide can be designed to include specific restriction enzyme cleavage site to facilitate ligation of the synthesized oligonucleotide into an expression vector.

For longer peptides, oligonucleotides can be prepared via solid phase synthesis and then the synthetic oligonucleotide sequences ligated together using various techniques. Recombinant techniques for the fabrication of whole synthetic genes are reviewed, for example, in Hughes et al., “Chapter Twelve—Gene Synthesis: Methods and Applications,” Methods in Enzymology 498:277-309 (2011), which is hereby incorporated by reference in its entirety.

Synthetic oligonucleotides of the present invention include both DNA and RNA, in both D and L enantiomeric forms, as well as derivatives thereof (including, but not limited to, 2′-fluoro-, 2′-amino, 2′O-methyl, 5′iodo-, and 5′-bromo-modified polynucleotides). Nucleic acids containing modified nucleotides (Kubik et al., “Isolation and Characterization of 2′fluoro-, 2′amino-, and 2′fluoro-amino-modified RNA Ligands or Human IFN-gamma that Inhibit Receptor Binding,” J. Immunol. 159:259-267 (1997); Pagratis et al., “Potent 2′-amino, and 2′-fluoro-2′-deoxy-ribonucleotide RNA Inhibitors of Keratinocyte Growth Factor,” Nat. Biotechnol. 15:68-73 (1997), each which is hereby incorporated by reference in its entirety) and the L-nucleic acids (sometimes termed Spiegelmers®), enantiomeric to natural D-nucleic acids (Klussmann et al., “Mirror-image RNA that Binds D-adenosine,” Nat. Biotechnol. 14:1112-1115 (1996) and Williams et al., “Bioactive and nuclease-resistant L-DNA Ligand of Vasopressin,” Proc. Natl. Acad. Sci. USA 94:11285-11290 (1997), each which is hereby incorporated by reference in its entirety), and non-natural bases are used to enhance biostability. In addition, the sugar-phosphate backbone can be replaced with a peptide backbone, forming a peptide nucleic acid (PNA), other natural or non-natural sugars can be used (e.g., 2′-deoxyribose sugars), or phosphothioate or phosphodithioate can be used instead of phosphodiester bonds. The use of locked nucleic acids (LNA) is also contemplated. These nucleic acid molecules can be used for multiple purposes, including application to plants or plants seeds as naked oligonucleotides or for in vitro translation of encoding oligonucleotides for production of the peptides of the present invention.

Once a suitable expression vector is selected, the desired nucleic acid sequences are cloned into the vector using standard cloning procedures in the art, as described by Sambrook et al., Molecular Cloning: A Laboratory Manual, Cold Springs Laboratory, Cold Springs Harbor, N.Y. (1989), or U.S. Pat. No. 4,237,224 to Cohen and Boyer, which are hereby incorporated by reference in their entirety. The vector is then introduced to a suitable host.

A variety of host-vector systems may be utilized to recombinantly express the peptides of the present invention. Primarily, the vector system must be compatible with the host used. Host-vector systems include, without limitation, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by Agrobacterium. The expression elements of these vectors vary in their strength and specificities. Depending upon the host-vector system utilized, any one of a number of suitable transcription and translation elements can be used to carry out this and other aspects of the present invention.

Purified peptides may be obtained by several methods. The peptide is preferably produced in purified form (preferably at least about 80% or 85% pure, more preferably at least about 90% or 95% pure) by conventional techniques. Depending on whether the recombinant host cell is made to secrete the peptide into growth medium (see U.S. Pat. No. 6,596,509 to Bauer et al., which is hereby incorporated by reference in its entirety), the peptide can be isolated and purified by centrifugation (to separate cellular components from supernatant containing the secreted peptide) followed by sequential ammonium sulfate precipitation of the supernatant. The fraction containing the peptide is subjected to gel filtration in an appropriately sized dextran or polyacrylamide column to separate the peptides from other proteins. If necessary, the peptide fraction may be further purified by HPLC.

Alternatively, if the peptide of interest is not secreted, it can be isolated from the recombinant cells using standard isolation and purification schemes. This includes disrupting the cells (e.g., by sonication, freezing, French press, etc.) and then recovering the peptide from the cellular debris. Purification can be achieved using the centrifugation, precipitation, and purification procedures described above. The use of purification tags, described above, can simplify this process.

In certain embodiments, purification is not required. Where purification is not performed, cell-free lysates can be recovered following centrifugation for removal of cellular debris. The resulting cell-free lysate can be treated with heat for a sufficient amount of time to deactivate any native proteases in the recovered fraction, e.g., 10 min at 100° C. If desired, one or more of biocidal agents, protease inhibitors, and non-ionic surfactants can be introduced to such a cell-free preparation (see U.S. Application Publ. No. 20100043095 to Wei, which is hereby incorporated by reference in its entirety).

Once the peptides of the present invention are recovered, they can be used to prepare a composition that includes a carrier, and one or more additives selected from the group consisting of a bacteriocidal or biocidal agent, a protease inhibitor, a non-ionic surfactant, a fertilizer, an herbicide, an insecticide, a fungicide, a nematicide, biological inoculants, plant regulators, and mixtures thereof.

In certain embodiments, the compositions include greater than about 1 nM of the peptide, greater than about 10 nM of the peptide, greater than about 20 nM of the peptide, greater than about 30 nM of the peptide, greater than about 40 nM of the peptide, greater than about 50 nM of the peptide, greater than about 60 nM of the peptide, greater than about 70 nM of the peptide, greater than 80 about nM of the peptide, greater than about 90 nM of the peptide, greater than about 100 nM of the peptide, greater than about 150 nM of the peptide, greater than about 200 nM of the peptide, or greater than about 250 nM of the peptide. In certain embodiments, the compositions include less than about 1 nM of the peptide. For example, certain peptides can be present at a concentration of less than about 2 ng/ml, less than about 1.75 ng/ml, less than about 1.5 ng/ml, less than about 1.25 ng/ml, less than about 1.0 ng/ml, less than about 0.75 ng/ml, less than about 0.5 ng/ml, less than about 0.25 ng/ml, or even less than about 0.1 ng/ml.

Suitable carriers include water, aqueous solutions optionally containing one or more co-solvents, slurries, and solid carrier particles. Exemplary solid carriers include mineral earths such as silicates, silica gels, talc, kaolins, limestone, lime, chalk, bole, loess, clays, dolomite, diatomaceous earth, calcium sulfate, magnesium sulfate, magnesium oxide, ground synthetic materials, and products of vegetable origin, such as cereal meal, tree bark meal, wood meal and nutshell meal, cellulose powders, starches and starch derivatives, as well as other mono-, di-, and poly-saccharides.

Suitable fertilizers include, without limitation, ammonium sulfate, ammonium phosphate, ammonium nitrate, ureas, and combinations thereof.

Suitable insecticides include, without limitation, members of the neonicotinoid class such as imidicloprid, clothianidin, and thiamethoxam; members of the organophosphate class such as chlorpyrifos and malathion; members of the pyrethroid class such as permethrin; other natural insecticides such as nicotine, nornicotine, and pyrethrins; members of the carbamate class such as aldicarb, carbofuran, and carbaryl; members of the macrocyclic lactone class such as various abamectin, avermectin, and ivermectin products; members of the diamide class such as chlorantraniliprole, cyantraniliprole, and flubendiamide; chitin synthesis inhibitors, particularly those of the benzoylurea class such as lufenuron and diflubenzuron; and any combination thereof, including combinations of two or more, three or more, or four or more insecticides. Additional insecticides are listed in the Compendium of Pesticide Common Names, which is database operated by Alan Wood and available in electronic form at the alanwood.net internet site.

Suitable fungicides include, without limitation, members of the strobilurin class such as azoxystrobin, pyraclostrobin, trifloxystrobin, picoxystrobin, and fluoxastrobin; members of the triazole class such as ipconazole, metconazole, tebuconazole, triticonazole, tetraconazole, difenoconazole, flutriafol, propiconazole and prothioconazole; members of the succinate dehydrogenase inhibitor class such as carboxin, fluxapyroxad, boscalid, sedaxane, and benzovindiflupyr (Solatenol™ by Syngenta); members of the phenylamide class such as metalaxyl, mefenoxam, benalaxyl, and oxadiyxl; members of the phenylpyrrole class such as fludioxonil; members of the phthalimide class such as captan; members of the dithiocarbamate class such as mancozeb and thiram; members of the benzimidazole class such as thiabendazole; fungicidal plant stimulators, such as acibenzolar-S-methyl; inorganic fungicides, such as copper compounds (notably copper hydroxide) and elemental sulfur; and any combination thereof, including combinations of two or more, three or more, or four or more fungicides. Additional fungicides are listed in the Compendium of Pesticide Common Names, which is a database operated by Alan Wood and available in electronic form at the alanwood.net internet site.

Suitable nematicides include, without limitation, chemicals of the carbamate class such as aldicarb, aldoxycarb, oxamyl, carbofuran, and cleothocarb; and chemicals of the organophosphate class such as thionazin, ethoprophos, fenamiphos, fensulfothion, terbufos, isazofos, and ebufos. Additional nematicides are listed in the Compendium of Pesticide Common Names, which is a database operated by Alan Wood and available in electronic form at the alanwood.net internet site.

Suitable bactericides include, without limitation, those based on dichlorophene and benzylalcohol hemi formal (Proxel® from ICI or Acticide® RS from Thor Chemie and Kathon® MK from Rohm & Haas) and isothiazolinone derivatives such as alkylisothiazolinones and benzisothiazolinones (Acticide® MBS from Thor Chemie; Proxel® GXL from ICI). Additional bactericides are listed in the Compendium of Pesticide Common Names, which is a database operated by Alan Wood and available in electronic form at the alanwood.net internet site.

Suitable inoculants include, without limitation, Bradyrhizobium spp., particularly Bradyrhizobium japonicum (BASF Vault® products), Bacillus subtilis, Bacillus firmus, Bacillus pumilis, Streptomyces lydicus, Trichoderma spp., Pasteuria spp., other cultures of rhizobial cells (BASF Nodulator® and Rhizo-Flo®), and any combination thereof, including combinations of two or more, three or more, or four or more inoculants. The inoculants can be recombinant in nature, as described hereinafter, to facilitate expression and optionally secretion of a polypeptide of the invention. Alternatively, these inoculants can be otherwise commercially available forms that are unable to express/secrete a polypeptide of the invention.

Plant regulators are chemical substances, whether natural or synthetic, that either stimulate or inhibit plant biochemical signaling. These are usually, but not exclusively, recognized by receptors on the surface of the cell, causing a cascade of reactions in the cell. Suitable plant regulators include, without limitation, ethephon; ethylene; salicylic acid; acetylsalicylic acid; jasmonic acid; methyl jasmonate; methyl dihydrojasmonate; chitin; chitosan; abscisic acid; any auxin compound or inhibitor, including but not limited to (4-chlorophenoxy)acetic acid, (2,4-dichlorophenoxy)acetic acid, and 2,3,5-triiodobenzoic acid; any cytokinin, including but not limited to kinetin and zeatin; gibberellins; brassinolide; and any combination thereof, including combinations of two or more, three or more, or four or more regulators.

Other suitable additives include buffering agents, wetting agents, coating agents, and abrading agents. These materials can be used to facilitate application of the compositions in accordance with the present invention. In addition, the compositions can be applied to plant seeds with other conventional seed formulation and treatment materials, including clays and polysaccharides.

Compositions or systems use for plant seed treatment include: one or more of the peptides of the present invention, preferably though not exclusively one of P12, P13-12, P13-14, P13-20, P13-3, P13-4, P13-5, P13-7, P13-s14, and P13-s15 (SEQ ID NOS: 5, 18, 20, 26, 9, 10, 11, 13, 83, and 84) in combination with one or more insecticides, nematicides, fungicides, other inoculants, or other plant regulators, including combinations of multiple insecticides, or multiple nematicides, multiple fungicides, multiple other inoculants, or multiple plant regulators. Suitable insecticides, nematicides, fungicides, inoculants, and plant regulators for these combination treatments include those identified above. These compositions are presented in the form of a single composition at the time of seed treatment. In contrast, a system used for seed treatment may involve multiple treatments, e.g., a composition containing the peptides is used in one treatment and a composition containing the one or more insecticides, nematicides, fungicides, plant regulators and/or bactericides, is used in a separate treatment. In the latter embodiment, both of these treatments are carried out at about the same time, i.e., before planting or at about the time of planting.

One such example includes one or more of peptides of the present invention, including (without limitation) one of P12, P13-12, P13-14, P13-20, P13-3, P13-4, P13-5, P13-7, P13-s14, and P13-s15 (SEQ ID NOS: 5, 18, 20, 26, 9, 10, 11, 13, 83, and 84), in combination with Poncho™ (clothianidin) available from Bayer Crop Science, Poncho™ VOTiVO (clothianidin and Bacillus firmus biological nematicide) available from Bayer Crop Science, and Gaucho™ (imidicloprid) available from Bayer Crop Science.

Another example includes one or more of peptides of the present invention, including (without limitation) one of P12, P13-12, P13-14, P13-20, P13-3, P13-4, P13-5, P13-7, P13-s14, and P13-s15 (SEQ ID NOS: 5, 18, 20, 26, 9, 10, 11, 13, 83, and 84), in combination with Cruiser™ (thiamethoxam) available from Syngenta, CruiserMaxx™ (thiamethoxam, mefenoxam, and fludioxynil) available from Syngenta, Cruiser Extreme™ (thiamethoxam, mefenoxam, fludioxynil, and azoxystrobin) available from Syngenta, Avicta™ (thiamethoxam and abamectin) available from Syngenta, and Avicta™ Complete (thiamethoxam, abamectin, and Clariva Complete™ which contains the Pasteuria nishizawae—Pn1 biological inoculant) available from Syngenta, and Avicta Complete™ Corn (thiamethoxam, mefenoxam, fludioxynil, azoxystrobin, thiabendazole and abamectin) available from Syngenta.

Another example includes one or more of peptides of the present invention, including (without limitation) one of P12, P13-12, P13-14, P13-20, P13-3, P13-4, P13-5, P13-7, P13-s14, and P13-s15 (SEQ ID NOS: 5, 18, 20, 26, 9, 10, 11, 13, 83, and 84), in combination with Vault Liquid plus Integral (Bradyrhizobium species and Bacillus subtilis strain MBI 600 inoculants) available from BASF, Vault NP (Bradyrhizobium japonicum inoculant) available from BASF, and Subtilex NG (Bacillus subtilis biological inoculant) available from BASF.

As an alternative to using peptides or compositions to apply the peptides of the present invention to plants, the use of recombinant host cells to deliver the peptide to the plant or plant seed, or the locus where the plant seed is planted in soil (and where the mature plant is grown), is also contemplated. Thus, a further aspect of the invention includes a recombinant host cell comprising a transgene that comprises a promoter-effective nucleic acid molecule operably coupled to a nucleic acid molecule that encodes a peptide or fusion polypeptide of the present invention, wherein the recombinant host cell is a microbe that imparts a first benefit to a plant grown in the presence of the recombinant microbe and the peptide or fusion polypeptide imparts a second benefit to the plant grown in the present of the recombinant microbe.

A “host cell” is a cell that contains a subject recombinant nucleic acid, either in the genome of the host cell or in an extrachromosomal vector that replicates autonomously from the genome of the host cell. A host cell may be any cell type.

In various embodiments, a host cell comprising a subject recombinant nucleic acid is provided. The host cell may be any cell type, but is preferably a microbe, e.g., a bacterial or fungal (such as a non-filamentous or filamentous fungal) host cell.

In certain embodiments, the microbe is a beneficial microbe that imparts a benefit to a plant grown in the presence of the microbe. A recombinant beneficial microbe also imparts a benefit to a plant grown in the presence of the microbe, but due to the presence of a recombinant polynucleotide the recombinant beneficial microbe also expresses a peptide or fusion polypeptide that imparts a second benefit to the plant grown in the presence of the recombinant microbe.

The term “filamentous fungi” refers to all filamentous forms of the subdivision Eumycotina (see Alexopoulos, C. J., INTRODUCTORY MYCOLOGY, Wiley, N.Y. (1962), which is hereby incorporated by reference in its entirety). These fungi are characterized by a vegetative mycelium with a cell wall composed of chitin, glucans, and other complex polysaccharides. The filamentous fungi of the present invention are morphologically, physiologically, and genetically distinct from yeasts. Vegetative growth by filamentous fungi is by hyphal elongation and carbon catabolism is obligatory aerobic.

In certain embodiments, the beneficial microbe is a bacterium.

Beneficial microbes execute a number of useful activities, reviewed in Glick, “Plant Growth-Promoting Bacteria: Mechanisms and Applications,” Scientifica, Article ID 963401 (2012), which is hereby incorporated by reference in its entirety. Beneficial microbes can provide nutrition to a plant. This may come in the form of amino acids and other nitrogen-containing compounds through the process of nitrogen fixation. Beneficial microbes may also liberate phosphate from inaccessible mineral deposits in the soil and make these available. For example, bacteria can synthesize siderophores which bind and solubilize inaccessible iron deposits. These iron-siderophore complexes can be absorbed by plants. Microbes can produce analogs of plant signaling hormones which stimulate growth and reduce stress signaling. Finally, beneficial microbes can compete with pathogenic organisms by removing resources including iron as well as synthesis of antibiotic compounds. Beneficial microbes may exhibit other behaviors and are not limited to the behaviors listed above. Beneficial organisms are classified as epiphytic (living on or near the surface of plant tissues) or endophytic (living within plant tissues).

Suitable beneficial bacterium include, without limitation, Pseudomonas (e.g., P. fluorescens, P. aureofaciens, P. chlororaphis, P. solanacearum, and P. syringae), Sphingomonas (e.g., S. phyllosphaerae, S. roseiflava, S. melonis, S. azotifigens, and S. mali) (see also Innerebner et al., “Protection of Arabidopsis thaliana Against Leaf-Pathogenic Pseudomonas syringae by Sphingomonas Strains in a Controlled Model System,” Appl. Environ. Microbiol. 77:3202-3210 (2011), which is hereby incorporated by reference in its entirety), Bacillus (B. firmus, B. licheniformis, B. megaterium, B. mucilaginous, B. pumilus, B. subtilis, and B. subtilis var. amyloliquefaciens), Streptomyces (e.g., S. griseoviridis and S. lydicus), Rhizobium (e.g., R. meliloti, R. trifolii, R. leguminosarum, R. phaseolin, R. lupine, and R. japonicum), Frankia (e.g., F. alni), and Azospirillum (e.g., A. brasilense and A. lipoferum).

Additional beneficial bacterium, include, without limitation, Agrobacterium radiobacter, Azotobacter chroococcum, Burkholderia cepacia, Delfitia acidovorans, Paenobacillus macerans, Pantoea agglomerans, and Serratia entomophilia.

In certain embodiments, the beneficial microbe may be a filamentous fungal host cell. In some embodiments, the host cell may be a cell of a strain that has a history of use for production of proteins that has GRAS status, i.e., a Generally Recognized as Safe, by the FDA.

In some embodiments, beneficial fungal microbes may be of a strain of Aspergillus niger which include ATCC 22342, ATCC 44733, ATCC 14331, ATCC 11490, NRRL 3112, and strains derived therefrom. In some embodiments, beneficial fungal microbes may be strains of Trichoderma (e.g. T. harzianum, T. viride, T. koningi, T. reesei and T. hamatum) which include functional equivalents of RL-P37 (Sheir-Neiss et al. (1984) Appl. Microbiol. Biotechnology 20:46-53, which is hereby incorporated by reference in its entirety). Other useful beneficial fungal microbes include, without limitation, NRRL 15709, ATCC 13631, ATCC 26921 (QM 9414) ATCC 32098, ATCC 32086, and ATCC 56765 (RUT-30). In some embodiments, beneficial fungal microbes may be strains of non-filamentous fungal yeasts, including, without limitation, strains of Rhodotorula (e.g., R. graminis WP1 and R. mucilaginosa) (see U.S. Pat. No. 8,728,781 and Xin et al., “Characterization of Three Endophytic, Indole-3-Acetic Acid-Producing Yeasts Occurring in Populus Trees,” Mycol. Res. 113:973-980 (2009), which are hereby incorporated by reference in their entirety).

In certain embodiments, the recombinant microbe is epiphytic. Such a microbe lives non-parasitically on the surface of the host plant tissues, including without limit, at the surface of leaves or near roots.

In other embodiments, the recombinant microbe is endophytic. Such a microbe lives at least part of its life-cycle non-parasitically within plant tissues, including without limit, within leaves, roots, and stems.

Peptide expression systems can be created using existing plasmid systems by one skilled in the art. One notable guideline is that regulation of peptide expression should be well controlled. High peptide concentrations detected by the plant will likely trigger an intense immune response with widespread cell death characteristic of the hypersensitive response. In contrast, lower peptide expression levels should stimulate the immunity while minimizing cell death. This effect may be further balanced by careful choice of secretion sequences. Expression of peptides in Pseudomonas fluorescens may be accomplished using the expression strains and tools described by Retallack et al., “Reliable protein production in a Pseudomonas fluorescens expression system,” Protein Expression and Purification 81:157-65 (2012), which is hereby incorporated by reference in its entirety. Expression of peptides in Bacillus subtilis can be accomplished through vectors utilizing a subtilisin (aprE) promoter system. This can optionally be augmented using signal peptides to direct secretion of the peptide outside of the microbe. These functions are implemented in the “Bacillus Subtilis Secretory Protein Expression System” manual available from Clontech. Expression of proteins in Streptomyces has been demonstrated using plasmids as described by Fernandez-Abalos et al., “Posttranslational processing of the xylanase Xys1L from Streptomyces halstedii JM8 is carried out by secreted serine proteases,” Microbiology 149:1623-32 (2003), which is hereby incorporated by reference in its entirety. Additional peptide expression systems can be produced by one skilled in the art.

The benefits attributable to the use of the recombinant beneficial microbe depend on the type of microbe and the plant peptide expressed thereby. In certain embodiments, the benefit attributable to the recombinant beneficial microbe is providing nutrients to a plant, producing plant hormone analogs that stimulate growth or reduce stress signaling, or competing with pathogenic organisms. In certain embodiments, the benefit attributable to the peptide or fusion polypeptide is improved disease resistance, growth enhancement, tolerance and resistance to biotic stressors, tolerance to abiotic stress, desiccation resistance for cuttings removed from ornamental plants, post-harvest disease resistance or desiccation resistance to fruit or vegetables harvested from plants, and/or improved longevity of fruit or vegetable ripeness for fruit or vegetables harvested from plants. Multiple different recombinant host cells can be used in combination.

Once engineered microbes are raised, e.g., in a fermentation apparatus, the engineered microbes can be recovered and then provided in either a dry composition or a liquid composition or suspension. For liquid compositions or suspensions, the microbes can be mixed in water, or a buffer solution, and applied as a spray treatment to the plants or the locus where plants are grown. Alternatively, the solution can be used as a seed treatment prior to planting the seeds. For dry compositions, the microbes can be dried with or without inert carrier particles, and the dry composition can be applied to seeds, the locus where seeds will be planted or plants are being grown, or directly to plants.

Colony forming units (c.f.u.) are used to quantify microbes. 1 c.f.u. of a microbe generates a single colony when spread onto a solid nutrient agar compatible with the organism and corresponds to one healthy, replication competent cell. In a dry powder formulation, the concentration of microbes can exceed 5×10¹⁰ cfu/gram of material. Suitable concentrations for a dry formulation include >10^{11,} >5×10¹⁰ ,>10¹⁰, >10⁹, >10⁸, 10⁷, or >10⁶ cfu/gram. Likewise, microbes can be provided as a liquid suspension. Suitable concentrations for a liquid formulation include >10¹⁰, >10⁹, >10⁸, >10⁷, >10⁶, >10⁵ cfu/ml.

Suitable carriers include water, aqueous solutions optionally containing one or more co-solvents, slurries, and solid carrier particles. Exemplary solid carriers include mineral earths such as silicates, silica gels, talc, kaolins, limestone, lime, chalk, bole, loess, clays, dolomite, diatomaceous earth, calcium sulfate, magnesium sulfate, magnesium oxide, ground synthetic materials, and products of vegetable origin, such as cereal meal, tree bark meal, wood meal and nutshell meal, cellulose powders, starches and starch derivatives, as well as other mono-, di-, and poly-saccharides. Exemplary aqueous solutions include those having pH 6-8, more preferably 6.5 to 7.5, containing a buffer matched to this range. Suitable buffers include, without limitation citrate, phosphate, carbonate, and HEPES. However, some microbes can persist in a spore form that is more resilient extremes of heat and pH as well as extended storage. Exemplary aqueous solutions compatible with this spore state include those having pH 3-8, more preferably 4.0-7.5, containing a buffer matched to this range. In addition to buffers described supra, suitable buffers include, without limitation, acetate, glutamate, and aspartate. The solution may optionally be supplemented with an enzymatic digest of proteins, yeast extract, and mineral nutrients, including but not limited to magnesium and iron.

Other suitable additives include buffering agents, wetting agents, coating agents, and abrading agents. These materials can be used to facilitate application of the compositions in accordance with the present invention.

For liquid compositions or suspensions, the microbes can be mixed in water, or a buffer solution, and applied as a spray or soaking treatment to the plant seeds, the plants or the locus where plants are grown. Alternatively, the solution can be applied prior to planting seeds at the locus, after planting seeds at the locus, prior to planting one or more seedlings at the locus, after planting one or more seedlings at the locus, or to the locus while plants are being grown at the locus.

For dry compositions, the microbes can be dried with or without inert carrier particles, and the dry composition can be applied to seeds, the locus where seeds will be planted or plants are being grown, or directly to plants.

As discussed hereinafter, the recombinant beneficial microbes can be used to impart multiple benefits to plants grown in the presence of the recombinant beneficial microbes. These uses involve application of the recombinant beneficial microbes directly to plant seeds, directly onto plants, or indirectly onto plants via application to the locus where seeds will be planted or plants are being grown. In these embodiments, the locus may include artificial or natural soil, a polymer growth medium, or a hydroponic growth medium. The soil can be present in any of a variety of environments including an open field, a partially covered field, a greenhouse, etc.

The present invention further relates to methods of imparting disease resistance to plants, enhancing plant growth, effecting pest control, imparting biotic or abiotic stress tolerance to plants, and/or modulating plant biochemical signaling. According to one embodiment, these methods involve applying an effective amount of an isolated peptide or fusion polypeptide of the invention, a recombinant host cell of the invention, or a composition of the invention to a plant or plant seed or the locus where the plant is growing or is expected to grow. As a consequence of such application, the peptide, fusion polypeptide, recombinant host cell, or composition contacts cells of the plant or plant seed, and induces in the plant or a plant grown from the plant seed disease resistance, growth enhancement, tolerance to biotic stress, tolerance to abiotic stress, or altered biochemical signaling. According to an alternative embodiment, the peptide, fusion polypeptide, recombinant host cell, or composition of the invention can be applied to plants such that seeds recovered from such plants themselves are able to impart disease resistance in plants, to enhance plant growth, to affect insect control, to impart tolerance to biotic or abiotic stress, and/or to modulate biochemical signaling, to modulate maturation.

In these embodiments, it is also possible to select plants or plant seeds or the locus to which the peptide, fusion polypeptide, recombinant host cell, or composition of the invention is applied. For example, for fields known to contain a high nematode content, the plants or plant seeds to be grown in such fields, or the fields (locus), can be selectively treated by applying the peptide, fusion polypeptide, recombinant host cell, or composition of the invention as described herein; whereas no such treatment may be necessary for plants or plant seeds grown in fields containing low nematode content. Similarly, for fields having reduced irrigation, the plants or plant seeds to be grown in such fields, or the fields (locus), can be selectively treated by applying the peptide, fusion polypeptide, recombinant host cell, or composition of the invention as described herein; whereas no such treatment may be necessary for plants or plant seeds grown in fields having adequate irrigation. Likewise, for fields prone to flooding, the plants or plant seeds to be grown in such fields, or the fields (locus), can be selectively treated by applying the peptide, fusion polypeptide, recombinant host cell, or composition as described herein; whereas no such treatment may be necessary for plants or plant seeds grown in fields that are not prone to flooding. As yet another example of such selection, for fields prone to insect attack at certain times of the growing season, the plants or plant seeds to be grown in such fields, or the fields (locus), can be selectively treated by applying the peptide, fusion polypeptide, recombinant host cell, or composition of the invention as described herein; whereas the same field may not be treated at ineffective times of the growing season or other fields that are not prone to such attack may go untreated. Such selection steps can be carried out when practicing each of the methods of use described herein, i.e., imparting disease resistance to plants, enhancing plant growth, effecting pest control (including insects and nematodes), imparting biotic or abiotic stress tolerance to plants, and/or modulating plant biochemical signaling.

As an alternative to applying an isolated peptide, fusion polypeptide, recombinant host cell, or composition containing the same to plants or plant seeds in order to impart disease resistance in plants, to effect plant growth, to control insects, to impart stress resistance and/or modulated biochemical signaling to the plants or plants grown from the seeds, transgenic plants or plant seeds can be utilized. When utilizing transgenic plants, this involves providing a transgenic plant transformed with a DNA molecule encoding a peptide of the invention and growing the plant under conditions effective to permit that DNA molecule to impart disease resistance to plants, to enhance plant growth, to control insects, to impart tolerance to biotic or abiotic stress, and/or to modulate biochemical signaling. Alternatively, a transgenic plant seed transformed with a DNA molecule encoding a peptide of the invention can be provided and planted in soil. A plant is then propagated from the planted seed under conditions effective to permit that DNA molecule to express the peptide and thereby impart disease resistance to the transgenic plant, to enhance plant growth, to control insects, to impart tolerance to biotic or abiotic stress, and/or to modulate biochemical signaling. This transgenic approach can be used in combination with the recombinant host cell, or topical application of the isolated peptide or composition.

The present invention further relates to methods of improving desiccation resistance for cuttings removed from ornamental plants, post-harvest disease resistance or desiccation resistance to fruit or vegetables harvested from plants, and/or improved longevity of fruit or vegetable ripeness for fruit or vegetables harvested from plants. These methods involve applying an effective amount of an isolated peptide, fusion polypeptide, recombinant host cell, or composition according to the present invention to a plant or the locus where the plant is growing. As a consequence of such application, the peptide contacts cells of the plant or plant seed, and induces desiccation resistance for cuttings removed from ornamental plants, post-harvest disease resistance or desiccation resistance to fruit or vegetables harvested from plants, and/or improved longevity of fruit or vegetable ripeness for fruit or vegetables harvested from plants. Alternatively, an effective amount of an isolated peptide, fusion polypeptide, recombinant host cell, or composition of the present invention or a composition according to the present invention can be applied to a harvested fruit or vegetable. As a consequence of such application, the peptide, fusion polypeptide, recombinant host cell, or composition contacts cells of the harvested fruit or vegetable, and induces post-harvest disease resistance or desiccation resistance to the treated fruit or vegetables, and/or improved longevity of fruit or vegetable ripeness for the treated fruit or vegetables.

As an alternative to applying an isolated peptide, fusion polypeptide, recombinant host cell, or composition containing the same to plants or plant seeds in order to induce desiccation resistance to cuttings removed from ornamental plants, post-harvest disease resistance or desiccation resistance to fruit or vegetables harvested from plants, and/or improved longevity of fruit or vegetable ripeness for fruit or vegetables harvested from plants, transgenic plants or plant seeds can be utilized. When utilizing transgenic plants, this involves providing a transgenic plant transformed with a DNA molecule encoding a peptide of the invention and growing the plant under conditions effective to permit that DNA molecule to induce desiccation resistance for cuttings removed from ornamental plants, post-harvest disease resistance or desiccation resistance to fruit or vegetables harvested from the transgenic plants, and/or improved longevity of fruit or vegetable ripeness for fruit or vegetables harvested from the transgenic plants. Alternatively, a transgenic plant seed transformed with a DNA molecule encoding a peptide of the invention can be provided and planted in soil. A plant is then propagated from the planted seed under conditions effective to permit that DNA molecule to express the peptide and thereby induce desiccation resistance for cuttings removed from ornamental plants, post-harvest disease resistance or desiccation resistance to fruit or vegetables harvested from the transgenic plants, and/or improved longevity of fruit or vegetable ripeness for fruit or vegetables harvested from the transgenic plants.

In these embodiments, it is also possible to select transgenic plants or plant seeds for carrying out the present invention. For example, for fields known to contain a high nematode content, the transgenic plants or plant seeds can be selectively grown in such fields; whereas non-transgenic plants or plant seeds can be grown in fields containing low nematode content. Similarly, for fields having reduced irrigation, the transgenic plants or plant seeds can be selectively grown in such fields; whereas non-transgenic plants or plant seeds can be grown in fields having adequate irrigation. Likewise, for fields prone to flooding, the transgenic plants or plant seeds can be grown in such fields; whereas non-transgenic plants or plant seeds can be grown in fields that are not prone to flooding. As yet another example of such selection, for fields prone to insect attack at certain times of the growing season, the transgenic plants or plant seeds can be selectively grown in such fields; whereas non-transgenic plants or plant seeds can be grown in fields that are not prone to such insect attack. Such selection steps can be carried out when practicing each of the methods of use described herein, i.e., imparting disease resistance to plants, enhancing plant growth, effecting pest control (including insects and nematodes), imparting biotic or abiotic stress tolerance to plants, and/or modulating plant biochemical signaling.

The present invention further relates to methods of improving desiccation resistance for cuttings removed from ornamental plants, post-harvest disease resistance or desiccation resistance to fruit or vegetables harvested from plants, and/or improved longevity of fruit or vegetable ripeness for fruit or vegetables harvested from plants. These methods involve applying an effective amount of a peptide, fusion polypeptide, recombinant host cell, or composition according to the present invention to a plant or the locus where the plant is growing. As a consequence of such application, the peptide, fusion polypeptide, recombinant host cell, or composition contacts cells of the plant or plant seed, and induces desiccation resistance for cuttings removed from ornamental plants, post-harvest disease resistance or desiccation resistance to fruit or vegetables harvested from plants, and/or improved longevity of fruit or vegetable ripeness for fruit or vegetables harvested from plants. Alternatively, an effective amount of an isolated peptide, fusion polypeptide, recombinant host cell, or composition according to the present invention can be applied to a harvested fruit or vegetable. As a consequence of such application, the peptide, fusion polypeptide, recombinant host cell, or composition contacts cells of the harvested fruit or vegetable, and induces post-harvest disease resistance or desiccation resistance to the treated fruit or vegetables, and/or improved longevity of fruit or vegetable ripeness for the treated fruit or vegetables.

In these embodiments, it is also possible to select plants, cuttings, fruits, vegetables, or the locus to which the isolated peptide or composition of the invention is applied. For example, for harvested cuttings or fruit or vegetables that are being shipped great distances or stored for long periods of time, then these can be selectively treated by applying the isolated peptide or composition of the invention as described herein; whereas harvested cuttings or fruit or vegetables that are being shipped locally and intended to be consumed without substantially periods of storage can be excluded from such treatment.

As an alternative to applying an isolated peptide, fusion polypeptide, recombinant host cell, or composition containing the same to plants or plant seeds in order to induce desiccation resistance to cuttings removed from ornamental plants, post-harvest disease resistance or desiccation resistance to fruit or vegetables harvested from plants, and/or improved longevity of fruit or vegetable ripeness for fruit or vegetables harvested from plants, transgenic plants or plant seeds can be utilized. When utilizing transgenic plants, this involves providing a transgenic plant transformed with a DNA molecule encoding a peptide of the invention and growing the plant under conditions effective to permit that DNA molecule to induce desiccation resistance for cuttings removed from ornamental plants, post-harvest disease resistance or desiccation resistance to fruit or vegetables harvested from the transgenic plants, and/or improved longevity of fruit or vegetable ripeness for fruit or vegetables harvested from the transgenic plants. Alternatively, a transgenic plant seed transformed with a DNA molecule encoding a peptide of the invention can be provided and planted in soil. A plant is then propagated from the planted seed under conditions effective to permit that DNA molecule to express the peptide and thereby induce desiccation resistance for cuttings removed from ornamental plants, post-harvest disease resistance or desiccation resistance to fruit or vegetables harvested from the transgenic plants, and/or improved longevity of fruit or vegetable ripeness for fruit or vegetables harvested from the transgenic plants.

In these embodiments, it is also possible to select transgenic plants or plant seeds for carrying out the present invention. For example, transgenic plants or plant seeds can be selected for growing when it is known that harvested cuttings or fruit or vegetables are intended to be shipped great distances or stored for long periods of time post-harvest; whereas non-transgenic plants or plant seeds can be selected for growing when it is known that harvested cuttings or fruit or vegetables are intended to be shipped locally and/or consumed without substantially periods of storage.

Suitable plants include dicots and monocots, including agricultural, silvicultural, ornamental and horticultural plants, whether in a natural or genetically modified form. Exemplary plants include, without limitation, alfalfa, apple, apricot, asparagus, avocados, bananas, barley, beans, beech (Fagus spec.), begonia, birch, blackberry, blueberry, cabbage, camphor, canola, carrot, castor oil plant, cherry, cinnamon, citrus, cocoa bean, coffee, corn, cotton, cucumber, cucurbit, eucalyptus, fir, flax, fodder beet, fuchsia, garlic, geranium, grapes, ground nut, hemp, hop, juneberry, juncea (Brassica juncea), jute, lentil, lettuce, linseed, melon, mustard, nectarine, oak, oats, oil palm, oil-seed rape, olive, onion, paprika, pea, peach, pear, pelargonium, peppers, petunia, pine (Pinus spec.), plum, poplar (Populus spec.), pome fruit, potato, rape, raspberry, rice, rubber tree, rye, sorghum, soybean, spinach, spruce, squash, strawberry, sugar beet, sugar cane, sunflower, tea, teak, tobacco, tomato, triticale, turf, watermelon, wheat and willow (Salix spec.), Arabidopsis thaliana, Saintpaulia, poinsettia, chrysanthemum, carnation, and zinnia.

With respect to modified biochemical signaling, this includes both enhancement of certain plant biochemical pathways and diminishment of certain other plant biochemical pathways. Biochemical signaling pathways that can be altered in accordance with the present invention include gene expression and protein production, production of metabolites, and production of signaling molecules/secondary metabolites. Exemplary biochemical signaling pathways and their modifications include, without limitation, induction of nitric oxide production, peroxide production, and other secondary metabolites; agonist of the ethylene signaling pathway and induction of ethylene-responsive gene expression (see Dong et al., Plant Phys. 136:3628-3638 (2004); Li et al., Planta 239:831-46 (2014); Chang et al., PLoS One 10,e0125498 (2015), each of which is hereby incorporated by reference in its entirety); agonist of the salicylic acid signaling pathway and induction of salicylic acid-responsive gene expression (see Dong et al., Plant J. 20:207-215 (1999), which is hereby incorporated by reference in its entirety); agonist of the abscisic acid pathway and induction of abscisic acid-responsive gene expression (see Dong et al., Planta 221: 313-327 (2005), which is hereby incorporated by reference in its entirety); agonist of the gibberellin signaling pathway and induction of gibberellin-responsive gene expression (see Li et al., Planta 239:831-46 (2014), which is hereby incorporated by reference in its entirety); antagonist of jasmonic acid signaling and inhibiting expression of jasmonic acid-responsive genes (see Dong et al., Plant Phys. 136:3628-3638 (2004), which is hereby incorporated by reference in its entirety); inducing protease inhibitor expression (see Laluk and Mengiste, Plant J. 1 68:480-494 (2011); Xia et al., Chin. Sci. Bull 56: 2351-2358 (2011), each of which is hereby incorporated by reference in its entirety); inducing reactive oxygen species production in plant tissues; inducing immune-related and antimicrobial peptide production, such as, without limitation, peroxidase, superoxide dismutase, chitinase, and β-1,3-glucanase (Wang et al., J. Agric. Food Chem. 59:12527-12533 (2011), which is hereby incorporated by reference in its entirety); and inducing expansin gene expression and production (see Li et al., Planta 239:831-46 (2014), which is hereby incorporated by reference in its entirety).

With respect to disease resistance, absolute immunity against infection may not be conferred, but the severity of the disease is reduced and symptom development is delayed. Lesion number, lesion size, and extent of sporulation of fungal pathogens are all decreased. This method of imparting disease resistance has the potential for treating previously untreatable diseases, treating diseases systemically which might not be treated separately due to cost, and avoiding the use of infectious agents or environmentally harmful materials.

The method of imparting pathogen resistance to plants in accordance with the present invention is useful in imparting resistance to a wide variety of pathogens including viruses, bacteria, and fungi. Resistance, inter alia, to the following viruses can be achieved by the method of the present invention: Tobacco mosaic virus and Tomato mosaic virus. Resistance, inter alia, to the following bacteria can also be imparted to plants in accordance with present invention: pathogenic Pseudomonas spp., pathogenic Erwinia spp., pathogenic Xanthomonas spp., and pathogenic Ralstonia spp. Plants can be made resistant, inter alia, to the following fungi by use of the method of the present invention: Fusarium spp. and Phytophthora spp.

With regard to the use of the peptides, fusion polypeptides, recombinant host cells, or compositions of the present invention to enhance plant growth, various forms of plant growth enhancement or promotion can be achieved. This can occur as early as when plant growth begins from seeds or later in the life of a plant. For example, plant growth according to the present invention encompasses greater yield, increased plant vigor, increased vigor of seedlings (i.e., post-germination), increased plant weight, increased biomass, increased number of flowers per plant, higher grain and/or fruit yield, increased quantity of seeds produced, increased percentage of seeds germinated, increased speed of germination, increased plant size, decreased plant height (for wheat), greater biomass, more and bigger fruit, earlier fruit coloration, earlier bud, fruit and plant maturation, more tillers or side shoots, larger leaves, delayed leaf senescence, increased shoot growth, increased root growth, altered root/shoot allocation, increased protein content, increased oil content, increased carbohydrate content, increased pigment content, increased chlorophyll content, increased total photosynthesis, increased photosynthesis efficiency, reduced respiration (lower O₂ usage), compensation for yield-reducing treatments, increased durability of stems (and resistance to stem lodging), increased durability of roots (and resistance to root lodging), better plant growth in low light conditions, and combinations thereof. As a result, the present invention provides significant economic benefit to growers. For example, early germination and early maturation permit crops to be grown in areas where short growing seasons would otherwise preclude their growth in that locale. Increased percentage of seed germination results in improved crop stands and more efficient seed use. Greater yield, increased size, and enhanced biomass production allow greater revenue generation from a given plot of land.

With regard to the use of the peptides or compositions of the present invention to control pests (including but not limited to insects and nematodes, which are biotic stressors), such pest control encompasses preventing pests from contacting plants to which the peptide or composition of the invention has been applied, preventing direct damage to plants by feeding injury, causing pests to depart from such plants, killing pests proximate to such plants, interfering with insect larval feeding on such plants, preventing pests from colonizing host plants, preventing colonizing insects from releasing phytotoxins, interfering with egg deposition on host plants, etc. The present invention also prevents subsequent disease damage to plants resulting from pest infection.

The present invention is effective against a wide variety of insects (biotic stressors). European corn borer is a major pest of corn (dent and sweet corn) but also feeds on over 200 plant species including green, wax, and lima beans and edible soybeans, peppers, potato, and tomato plus many weed species. Additional insect larval feeding pests which damage a wide variety of vegetable crops include the following: beet armyworm, cabbage looper, corn ear worm, fall armyworm, diamondback moth, cabbage root maggot, onion maggot, seed corn maggot, pickleworm (melonworm), pepper maggot, and tomato pinworm. Collectively, this group of insect pests represents the most economically important group of pests for vegetable production worldwide. The present invention is also effective against nematodes, another class of economically important biotic stressors. Soybean Cyst Nematode (Heterodera glycines) is a major pest of soybeans. Reniform Nematode (Rotylenchulus reniformis) is a major pest of cotton as can parasitize additional crop species, notably soy and corn. Additional nematode pests include the root knot nematodes of the genus Meloidogyne (particularly in cotton, wheat, and barley), cereal cyst nematodes of the genus Heterodera (particularly in soy, wheat, and barley), root lesion nematodes of the genus Pratylenchus, seed gall nematodes of the genus Anguina (particularly in wheat, barley, and rye), and stem nematodes of the genus Ditylenchus. Other biotic stressors include arachnids, weeds, and combinations thereof.

With regard to the use of the peptides, fusion polypeptides, recombinant host cells, or compositions of the present invention to impart abiotic stress resistance to plants, such abiotic stress encompasses any environmental factor having an adverse effect on plant physiology and development. Examples of such environmental stress include climate-related stress (e.g., drought, flood, frost, cold temperature, high temperature, excessive light, and insufficient light), air pollution stress (e.g., carbon dioxide, carbon monoxide, sulfur dioxide, NO_{R}, hydrocarbons, ozone, ultraviolet radiation, acidic rain), chemical (e.g., insecticides, fungicides, herbicides, heavy metals), nutritional stress (e.g., over- or under-abundance of fertilizer, micronutrients, macronutrients, particularly potassium, nitrogen derivatives, and phosphorus derivatives), and improved healing response to wounding. Use of peptides, fusion polypeptides, recombinant host cells, or compositions of the present invention imparts resistance to plants against such forms of environmental stress.

A further aspect of the present invention relates to the use of the peptides of the present invention as a safener in combination with one or more of the active agents (i.e., in a composition or in separate compositions) for the control of aquatic weeds in a body of water as described in U.S. Publ. No. 20150218099 to Mann, which is hereby incorporated by reference in its entirety.

Yet another aspect of the present invention relates to the use of the peptides of the present invention as a plant strengthener in a composition for application to plants grown under conditions of reduced water irrigation, which composition also includes at least one antioxidant and at least one radiation manager, and optionally at least one plant growth regulator, as described in U.S. Publ. No. 20130116119 to Rees et al., which is hereby incorporated by reference in its entirety.

The methods of the present invention involving application of the peptide, fusion polypeptide, or composition can be carried out through a variety of procedures when all or part of the plant is treated, including leaves, stems, roots, propagules (e.g., cuttings), fruit, etc. This may (but need not) involve infiltration of the peptide into the plant. Suitable application methods include high or low pressure spraying, injection, and leaf abrasion proximate to when peptide application takes place. When treating plant seeds, in accordance with the application embodiment of the present invention, the hypersensitive response elicitor peptide or fusion polypeptide can be applied by low or high pressure spraying, coating, immersion (e.g., soaking), or injection. Other suitable application procedures can be envisioned by those skilled in the art provided they are able to effect contact of the hypersensitive response elicitor fusion polypeptide or protein with cells of the plant or plant seed. Once treated with the peptides or compositions of the present invention, the seeds can be planted in natural or artificial soil and cultivated using conventional procedures to produce plants. After plants have been propagated from seeds treated in accordance with the present invention, the plants may be treated with one or more applications of the peptides or compositions of the invention to impart disease resistance to plants, to enhance plant growth, to control insects on the plants, to impart biotic or abiotic stress tolerance, to improve desiccation resistance of removed cuttings, to impart post-harvest disease resistance or desiccation resistance to harvested fruit or vegetables, and/or improved longevity of fruit or vegetable ripeness for harvested fruit or vegetables.

Where the peptides are applied in the form of a recombinant host cell, these microbes can be applied in the form of an aqueous solution comprising a suspension of such beneficial microbes, which is then applied to the plant by spraying, coating, or immersion as described above. When treating plant seeds, in accordance with the application embodiment of the present invention, the microbes can be applied by low or high pressure spraying, coating, immersion (e.g., soaking), or injection. Other suitable application procedures can be envisioned by those skilled in the art provided they are able to effect contact of the beneficial microbes with cells of the plant or plant seed. In accordance with the application embodiment of the present invention, the beneficial microbes can be applied to plants or plant seeds in dry form. By way of example, dry application of microbes can be accomplished using bacterial or fungal products such as Kodiak® HB, available from Chemtura, and T-22™ HC, available from BioWorks. Once treated with the microbes of the present invention, the seeds can be planted in natural or artificial soil and cultivated using conventional procedures to produce plants. After plants have been propagated from seeds treated in accordance with the present invention, the plants may be treated with one or more applications of the recombinant host cells of the invention or the peptides, fusion polypeptides, or compositions of the invention, to impart disease resistance to plants, to enhance plant growth, to control insects on the plants, to impart biotic or abiotic stress tolerance, to improve desiccation resistance of removed cuttings, to impart post-harvest disease resistance or desiccation resistance to harvested fruit or vegetables, and/or improved longevity of fruit or vegetable ripeness for harvested fruit or vegetables.

The peptides, fusion polypeptides, recombinant host cells, or compositions of the invention can be applied to plants or plant seeds in accordance with the present invention alone or in a mixture with other materials. Alternatively, the peptides, fusion polypeptides, recombinant host cells, or compositions can be applied separately to plants with other materials being applied at different times.

In the alternative embodiment of the present invention involving the use of transgenic plants and transgenic seeds, a peptide of the invention need not be applied topically to the plants or seeds. Instead, transgenic plants transformed with a DNA molecule encoding a peptide of the invention are produced according to procedures well known in the art. A vector suitable for expression in plants (i.e., containing translation and transcription control sequences operable in plants) can be microinjected directly into plant cells by use of micropipettes to transfer mechanically the recombinant DNA (Crossway, Mol. Gen. Genetics, 202:179-85 (1985), which is hereby incorporated by reference in its entirety). The genetic material may also be transferred into the plant cell using polyethylene glycol (Krens, et al., Nature, 296:72-74 (1982), which is hereby incorporated by reference in its entirety).

Another approach to transforming plant cells with a gene encoding the peptide of the invention is particle bombardment (also known as biolistic transformation) of the host cell. This can be accomplished in one of several ways. The first involves propelling inert or biologically active particles at cells. This technique is disclosed in U.S. Pat. Nos. 4,945,050, 5,036,006, and 5,100,792, all to Sanford et al., which are hereby incorporated by reference. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector containing the heterologous DNA. Alternatively, the target cell can be surrounded by the vector so that the vector is carried into the cell by the wake of the particle. Biologically active particles (e.g., dried bacterial cells containing the vector and heterologous DNA) can also be propelled into plant cells.

Yet another method of introduction is fusion of protoplasts with other entities, either minicells, cells, lysosomes or other fusible lipid-surfaced bodies (Fraley, et al., Proc. Natl. Acad. Sci. USA, 79:1859-63 (1982), which is hereby incorporated by reference in its entirety) The DNA molecule may also be introduced into the plant cells by electroporation (Fromm et al., Proc. Natl. Acad. Sci. USA, 82:5824 (1985), which is hereby incorporated by reference in its entirety) In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide, and regenerate.

Another method of introducing the DNA molecule into plant cells is to infect a plant cell with Agrobacterium tumefaciens or A. rhizogenes previously transformed with the gene. Under appropriate conditions known in the art, the transformed plant cells are grown to form shoots or roots, and develop further into plants. Generally, this procedure involves inoculating the plant tissue with a suspension of bacteria and incubating the tissue for 48 to 72 hours on regeneration medium without antibiotics at 25-28° C. Agrobacterium is a representative genus of the gram-negative family Rhizobiaceae. Its species are responsible for crown gall (A. tumefaciens) and hairy root disease (A. rhizogenes). The plant cells in crown gall tumors and hairy roots are induced to produce amino acid derivatives known as opines, which are catabolized only by the bacteria. The bacterial genes responsible for expression of opines are a convenient source of control elements for chimeric expression cassettes. In addition, assaying for the presence of opines can be used to identify transformed tissue. Heterologous genetic sequences can be introduced into appropriate plant cells, by means of the Ti plasmid of A. tumefaciens or the Ri plasmid of A. rhizogenes. The Ti or Ri plasmid is transmitted to plant cells on infection by Agrobacterium and is stably integrated into the plant genome (J. Schell, Science, 237:1176-83 (1987), which is hereby incorporated by reference in its entirety).

After transformation, the transformed plant cells must be regenerated. Plant regeneration from cultured protoplasts is described in Evans et al., Handbook of Plant Cell Cultures, Vol. 1: (MacMillan Publishing Co., New York, 1983); and Nasil I. R. (ed.), Cell Culture and Somatic Cell Genetics of Plants, Acad. Press, Orlando, Vol. 1, 1984, and Vol. I11 (1986), which are hereby incorporated by reference in their entirety.

It is known that practically all plants can be regenerated from cultured cells or tissues. Means for regeneration varies from species to species of plants, but generally a suspension of transformed protoplasts or a petri plate containing transformed explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

After the expression cassette is stably incorporated in transgenic plants, it can be transferred to other plants by sexual crossing. Any of a number of standard breeding techniques can be used, depending upon the species to be crossed.

Once transgenic plants of this type are produced, the plants themselves can be cultivated in accordance with conventional procedure with the presence of the gene encoding the hypersensitive response elicitor resulting in disease resistance, enhanced plant growth, control of insects on the plant, abiotic or biotic stress tolerance, improved desiccation resistance of removed cuttings, post-harvest disease resistance or desiccation resistance in harvested fruit or vegetables, and/or improved longevity of fruit or vegetable ripeness for harvested fruit or vegetables.

Alternatively, transgenic seeds are recovered from the transgenic plants. These seeds can then be planted in the soil and cultivated using conventional procedures to produce transgenic plants. The transgenic plants are propagated from the planted transgenic seeds under conditions effective to impart disease resistance to plants, to enhance plant growth, to control insects, to impart abiotic or biotic stress tolerance, to improve desiccation resistance of removed cuttings, to impart post-harvest disease resistance or desiccation resistance in harvested fruit or vegetables, and/or to impart improved longevity of fruit or vegetable ripeness for harvested fruit or vegetables.

When transgenic plants and plant seeds are used in accordance with the present invention, they additionally can be treated with the same materials as are used to treat the plants and seeds to which a peptide, fusion polypeptide, recombinant host cell, or composition of the invention is applied. These other materials, including peptides, fusion polypeptides, recombinant host cells, or compositions of the invention, can be applied to the transgenic plants and plant seeds by the above-noted procedures, including high or low pressure spraying, injection, coating, and immersion. Similarly, after plants have been propagated from the transgenic plant seeds, the plants may be treated with one or more applications of the peptides, fusion polypeptides, recombinant host cells, or compositions of the invention to impart disease resistance, enhance growth, control insects, abiotic or biotic stress tolerance, desiccation resistance of removed cuttings, post-harvest disease resistance or desiccation resistance in harvested fruit or vegetables, and/or improved longevity of fruit or vegetable ripeness for harvested fruit or vegetables.

Such transgenic plants may also be treated with conventional plant treatment agents, e.g., bacteriocidal or biocidal agents, protease inhibitors, non-ionic surfactants, fertilizers, herbicides, insecticides, fungicides, nematicides, biological inoculants, plant regulators, and mixtures thereof, as described above.

### EXAMPLES

The following examples are provided to illustrate embodiments of the present invention but are by no means intended to limit its scope.

### Example 1

### Induction of Resistance to Tobacco Mosaic Virus

Peptides were tested for the induction of resistance to tobacco mosaic virus (TMV) in tobacco. Briefly, three tobacco plants at 6-8 weeks old were selected per group (samples and controls). The bottom-most leaf of the plant was covered and the plant was sprayed with a solution of water (untreated control—UTC), peptide, or Proact (positive control). The spray was applied until the leaves were fully wetted, indicated by liquid dripping from the leaves. The plants were then allowed to dry and the leaf covering was removed.

Three days post-treatment, the previously-covered leaf and a leaf on the opposite side of the plant were then lightly dusted with diatomaceous earth and 20 ul of a 1.7 μg/ml solution of purified tobacco mosaic virus was applied. The TMV solution was then spread across the leaf surface by lightly rubbing solution and the diatomaceous earth across the surface of the leaves. Two minutes after inoculation, the diatomaceous earth was rinsed off the leaves with water. 3 days after TMV inoculation, the leaves were scored based on the number of TMV lesions observed. The leaf was also scored for signs of the hypersensitive response, including yellowing and wilting of the affected leaves.

Effectiveness described in Table 5 refers to the % decline in TMV lesions on treated vs UTC plants. A reduction of TMV on covered leaves indicates a systemic immune response in the plant while reduction on uncovered leaves indicates a local response. Asterisks indicate that the P-value derived from a T-test was <0.05.

TABLE 5
Summary of TMV Resistance

| | | | Effectiveness | Effectiveness |
|---|---|---|---|---|
| | SEQ ID | Concentration | Uncovered | Covered |
| Peptide | NO: | (μg/ml) | (%) | (%) |
| P12 | 5 | 10 | 53* | 66* |
| P13-10 | 16 | 20 | 90.6* | 92.4 |
| P13-11 | 17 | 20 | 74.9* | 73.1 |
| P13-12 | 18 | 20 | 44.1 | 37.9 |
| P13-13 | 19 | 20 | 36.6 | 38.5 |

### Example 2

### Drought Resistance in Corn

The effectiveness of peptide treatment in reducing drought stress was assessed in corn. 3.5 inch pots were filled with Sunshine #1 soil (SunGro Horticulture), fertilized with a 20-10-20 mixture. The soil was soaked and drained overnight. Seeds (either corn or soy, manually inspected to ensure uniform seed size) were planted at a depth of 1 inch for germination. Plants were grown in a greenhouse under 16 hour light days at >70° F. and 8 hour dark nights at >65° F. Prior to drought conditions, the plants were well-watered.

When the plants reached the V1 stage, plants were culled to achieve a uniform height (abnormally large and small plants were removed). Plants were then randomly assigned to control (spray without peptide) or treatment (spray with peptide) groups and heights were measured. Peptide was made up in a solution of 0.2 μg/ml or 2μg/ml in distilled water+0.01% Tween-20, and applied as a fine mist from a spray bottle until the solution drips from the leaves. After the peptide solutions were dried, the plants were again randomized in a randomized complete block design. Drought stress was initiated after the peptide treatment. This was caused by maintaining the water level at 25-50% of the maximum capacity for water (capacity was decided as the weight of the pot filled with saturated soil minus the weight of the filled pot prior to adding water).

The drought test phase ended after 2-3 weeks. At that time, the plant height was again measured and the growth rate was calculated as the difference between this and the previously-recorded height. The above-ground part portions of the plants were harvested and weighed to obtain fresh weight. The above-ground portion was also dried in an oven at 70° C. for 72 hours to obtain dry weight. All calculations were compared with matched untreated control plants.

The drought testing procedure was carried out in corn using a treatment of P12, P13-10, P13-11, P13-12, P13-14, P13-4, and P13-15 (SEQ ID NOs: 5, 16, 17, 18, 20, 10, and 21). Results are shown in Table 6. Asterisks indicate statistical significance by P-value (*: P<0.1 and **: P<0.05)

TABLE 6
Summary of Drought Resistance

| | | | Dry Weight | Fresh Weight |
|---|---|---|---|---|
| | SEQ ID | Concentration | Increase | Increase |
| Peptide | NO: | (μg/ml) | (%) | (%) |
| P12 | 5 | 2 | 6.28** | 4.87* |
| P13-10 | 16 | 2 | 7.56* | 4.44 |
| P13-11 | 17 | 0.2 | 4.91* | 3.81* |
| P13-12 | 18 | 0.2 | 7.31* | 3.93* |
| P13-14 | 20 | 2 | 2.48 | 4.36* |
| P13-4 | 10 | 2.0 | 0.06 | −2.77 |
| P13-15 | 21 | 0.2 | 6.21 | 2.92 |

### Example 3

### Drought Resistance in Soy

The effectiveness of peptide treatment in reducing drought stress was assessed in corn. 3.5 inch pots were filled with Sunshine #1 soil (SunGro Horticulture), fertilized with a 20-10-20 mixture. The soil was soaked and drained overnight. Seeds (soy, manually inspected to ensure uniform seed size) were planted at a depth of half inch for germination. Plants were grown in a greenhouse under 16 hour light days at >70° F. and 8 hour dark nights at >65° F. Prior to drought conditions, the plants were well-watered.

When the plants reached the growth stage when first trifoliate expanded, plants were culled to achieve a uniform height (abnormally large and small plants were removed). Plants were then randomly assigned to control (spray without peptide) or treatment (spray with peptide) groups and heights were measured. Peptide was made up in a solution of 0.2 μg/ml or 2μg/ml in distilled water+0.04% Tween-20, and applied as a fine mist from a spray bottle until the solution drips from the leaves. After the peptide solutions were dried, the plants were again randomized in a randomized complete block design. Cyclic drought stress was initiated after the peptide treatment. Plants were subjected to at least three drought cycles of drought stress (3-5 days of withholding water and 1 day irrigated with saturating amount of water) before harvesting.

The drought test phase ended after 2-3 weeks. At that time, the plant height was again measured and the growth rate was calculated as the difference between this and the previously-recorded height. The above-ground part portions of the plants were harvested and weighed to obtain fresh weight. The above-ground portion was also dried in an oven at 70° C. for 72 hours to obtain dry weight. All calculations were compared with matched untreated control plants.

The drought testing procedure was carried out in soy using a treatment of P12-2, P13-4, P13-5, P13-7, P13-3, P13-14, P13-2, and P13-15 (SEQ ID NOs: 6, 10, 11, 13, 9, 20, 8, and 21). Results are shown in Table 7. Asterisks indicate statistical significance by P-value (*: P<0.1 and **: P<0.05)

TABLE 7
Summary of Drought Resistance

| | | | Dry Weight | Fresh Weight |
|---|---|---|---|---|
| | SEQ ID | Concentration | Increase | Increase |
| Peptide | NO: | (μg/ml) | (%) | (%) |
| P12-2 | 6 | 2 | 3.55 | 7.83** |
| P13-4 | 10 | 0.2 | 4.45* | 7.75** |
| P13-5 | 11 | 2 | 4.36 | 9.73** |
| P13-7 | 13 | 2 | 4.50* | 5.80* |
| P13-3 | 9 | 2 | 2.54 | 5.90** |
| P13-14 | 20 | 0.2 | −3.68** | −2.86 |
| P13-2 | 8 | 0.2 | 8.70** | 7.84** |
| P13-2 | 8 | 2.0 | 14.54** | 15.56** |
| P13-3 | 9 | 0.2 | 8.47** | 11.43** |
| P13-3 | 9 | 2.0 | 6.07** | 13.61** |
| P13-5 | 11 | 0.2 | 6.93** | 12.23** |
| P13-15 | 21 | 2.0 | 2.07 | 5.38* |

These results confirm that a smaller consensus sequence affords peptides that induce active plant responses, where three additional residues at the N-terminal end of any one of SEQ ID NOS: 1-3, and one additional amino acid residue at the C-terminal end of any one of SEQ ID NOS: 1-3 are sufficient for drought resistance activity.

### Example 4

### Drought Resistance in Seed-Coated Corn

The effectiveness of peptide treatment in reducing the effects of drought stress was assessed in corn using a seed treatment strategy. This strategy largely mirrors example 2, except that the seeds were coated with a peptide formulation rather than a foliar spray application.

Seeds were first sieved using mesh screens to a uniform size (21-23/64 inches). The seeds were then coated with a mixture of peptide, Unicoat™ seed coat polymer, and a minimal volume of water in a Hege 11 Liquid Seed Treater (Wintersteiger) according to manufacturer recommendations. Seeds were coated with one of the following: (i) 0.12 μg peptide/seed, (ii) 1.05 peptide/seed, or (iii) a ‘mock’ treatment that included no peptide. The amount of peptide per seed assumes complete transfer of the peptide to seed surfaces in the seed coating chamber. Losses during coating were not considered.

3.5 inch pots were filled (approximately 0.2 L per pot) with Sunshine #1 mix (SunGro Horticulture), fertilized with 14-14-14 N,P,K Osmocote (Everris), and mixed for 20 minutes using a soil mixer M-5. Seeds were planted at a depth of 0.5 inch for germination. The pots were immediately irrigated by submergence then drained after about an hour. For each treatment group, 24 replicate seeds were planted. Plants were grown in a greenhouse under 16 hour light days at ˜75° F. and 8 hour dark nights at ˜65° F. Prior to drought conditions, the plants were well-watered.

Three days after planting, the plants were randomized using a complete block design and subjected to a cyclic drought stress. Drought stress was imposed by withholding water for 2-3 days until the majority of plants were under medium to severe water stress. The severity of the stress was determined by the weight of the pot and visual determination of leaf curling. Once drought stress was attained, the pots were watered to full saturation. Full Saturation (100% pot capacity) is defined as the weight of the pot filled with water-saturated soil minus the weight of the filled pot prior to adding water. Drought stress was imposed for a total of 18 days.

At the end of the experiment (18 days after initiation of drought stress), the plant was then harvested for fresh and dry weights. Individual plants were cut near the soil surface and weighed immediately (fresh weight). The plants were then placed into labeled brown paper bags and dried in a 70° C. oven for 72 hours. A dry weight was then obtained.

The following calculation was performed on the collected data: Fresh weight at harvest and dry weight at harvest for the plants grown from peptide-treated seeds were expressed as a % change relative to the ‘mock’ treated plants. Results are shown in Table 8 below.

TABLE 8
Summary of Drought Resistance Following Corn Seed Treatment

| | SEQ ID | Application Rate | % Change | |
|---|---|---|---|---|
| Peptide | NO: | (μg/seed) | Dry Weight | Fresh Weight |
| P13-2 | 8 | 1.05 | 0.38 | 3.67 |
| P13-3 | 9 | 0.12 | 2.81 | 1.49 |
| P13-5 | 11 | 1.05 | 5.07* | 5.66** |
| P13-7 | 13 | 0.12 | 4.77** | 5.6* |
| P13-14 | 20 | 0.12 | 3.77 | 9.11** |
| P13-14 | 20 | 1.05 | 7.92** | 11.83** |
| P13-17 | 23 | 1.05 | 1.09 | 1.51 |
| P13-20 | 26 | 1.05 | 3.23 | 6.87** |
| P13-22 | 28 | 1.05 | 1.66 | 3.41 |
| P13-23 | 29 | 0.12 | 0.94 | 4.89** |
| P13-23 | 29 | 1.05 | 2.16 | 5.54** |
| P13-25 | 31 | 1.05 | 3.01 | 2.63 |
| P13-26 | 32 | 0.12 | 4.74** | 3.77* |
| P13-26 | 32 | 1.05 | 5.92** | 4.23* |
| P13-28 | 34 | 0.12 | 8.52** | 9.99** |
| P13-28 | 34 | 1.05 | 6.04** | 8.18** |
| P13-30 | 36 | 0.12 | 3.01 | 6.19* |
| P13-30 | 36 | 1.05 | 2.73 | 8.68** |
| P13-33 | 39 | 1.05 | 2.59 | −1.28 |
| P13-35 | 41 | 0.12 | 5.68** | 2.45 |
| P13-36 | 42 | 0.12 | 5.93** | 10.60** |
| P13-36 | 42 | 1.05 | 7.95** | 12.38** |
| P13-37 | 43 | 1.05 | 5.53** | 7.67** |
| P13-39 | 61 | 1.05 | 3.10 | 5.09** |
| P13-40 | 62 | 1.05 | 0.76 | 5.86* |
| P13-43 | 65 | 0.12 | −0.75 | 6.2* |
| P13-43 | 65 | 1.05 | 6.73** | 5.04 |
| P13-44 | 66 | 0.12 | 2.73 | 7.76** |
| P13-44 | 66 | 1.05 | 3.44 | 9.15** |
| P13-45 | 67 | 0.12 | 6.05* | 8.54** |
| P13-47 | 69 | 1.05 | −1.34 | −3.42 |
| P13-48 | 70 | 1.05 | −1.83 | −1.65 |
| P13-49 | 71 | 0.12 | 0.79 | −4.24 |
| P13-50 | 72 | 0.12 | 0.49 | −3.95 |
| P13-51 | 73 | 1.05 | 0.64 | −4.70 |
| P13-52 | 74 | 1.05 | 4.49** | −5.08* |
| P13-53 | 75 | 0.12 | 0.48 | −2.19 |
| P13-54 | 76 | 0.12 | −1.38 | −4.51* |
| P13-55 | 77 | 0.12 | 0.81 | −3.84 |
| P13-56 | 78 | 0.12 | 0.44 | 1.91 |
| P13-57 | 79 | 1.05 | −0.68 | 1.90 |
| P13-58 | 80 | 0.12 | 2.83 | −4.07 |

Development of consensus sequences and preferred embodiments, described supra in the detailed description, were informed by the results described above and our experience with other harpin-derived bioactive peptides, as described in PCT Application Publication Nos. WO2016/054310 and WO2016/054342, which are hereby incorporated by reference in their entirety. Briefly, our prior results indicated that the identity and placement of hydrophobic amino acids are most crucial for activity. Mutation of hydrophobic residues tends to reduce or eliminate activity. In general, the following mutations have been well-tolerated, mutation of M to L, and mutation between Ito L and L to I, and in some cases to V and F.

Several of the results support the conclusion that methionine amino acids can be replaced with leucine residues, including p13-5 (SEQ ID NO: 11), p13-7 (SEQ ID NO: 13), p13-23 (SEQ ID NO: 29), and p13-35 (SEQ ID NO: 41). These substitutions increase the stability of the peptide by eliminating methionine oxidation. The positive results for p13-30 (SEQ ID NO: 36) show that the two isoleucine residues can be mutated to leucine. However, the negative result for p13-33 (SEQ ID NO: 39) suggests that both phenylalanine residues should not be mutated. The positive result for dry weight using p13-52 (SEQ ID NO: 74) suggests that the phenylalanine at position 11 in SEQ ID NO: 1 is dispensable for drought tolerance activity when additional sequences are present. All other hydrophobic residues appear to be necessary.

In contrast, the identity of hydrophilic amino acids tends to be less important to peptide activity. In our experience, mutation of these residues, particularly to other hydrophilic amino acids (R, K, D, E, Q, N, H, S, T, G, P) as well as A, does not generally cause a loss of activity. The experimental results in Example 2 and this example (supra) support the belief of flexibility in substituting hydrophilic residues. In particular, P13-7 (SEQ ID NO: 13), P13-26 (SEQ ID NO: 32), P13-28 (SEQ ID NO: 34), p13-35 (SEQ ID NO: 41), and p13-37 (SEQ ID NO: 43) show that all of the hydrophilic amino acids within the sequence can be safely mutated to glutamate with preservation of activity. Further, the substitution of hydrophilic residues for glutamate surprisingly allows for a shorter active sequence; p13-35 (SEQ ID NO: 41) is only 19 amino acids—the shortest sequence that provides drought tolerance in corn. In contrast, if sequences more similar to wild-type are used, a longer sequence affords activity, e.g. p13-12 (SEQ ID NO: 18), p13-20 (SEQ ID NO: 26), or p13-23 (SEQ ID NO: 29). In some cases, glutamate residues added to the N-terminal and/or C-terminal ends of the consensus sequence (SEQ ID 1, 2, or 3) also supported corn drought tolerance, e.g. P13-5 (SEQ ID NO: 11), P13-26 (SEQ ID NO: 32), and P13-36 (SEQ ID NO: 42). Thus, the core consensus sequence (SEQ ID 1, 2, or 3) is sufficient to confer drought tolerance activity in corn, depending on the identity of the internal hydrophilic residues or N- and C-terminal extensions. Glutamate within the consensus sequence (at least 5 residues) confers drought tolerance activity. Alternatively, adding a combination of at least 4 glutamate residues to the N- and/or C-terminii also confers drought tolerance activity. Otherwise, addition of at least 6 residues to the N-terminus and at least 4 residues to the C-terminus affords activity in corn.

### Example 5

### Drought Resistance in Seed-Treated Soy

The effectiveness of peptide treatment in reducing drought stress was assessed in soy using a seed treatment strategy. This strategy largely mirrors examples 3 and 4.

Seeds were first sieved using mesh screens to a uniform size (14-17 mesh). The seeds were then coated with a mixture of peptide and Unicoat™ seed coat polymer in a Hege 11 Liquid Seed Treater (Wintersteiger) according to manufacturer recommendations. In general, seeds were coated with one of the following: (i) 0.12 μg peptide/seed, (ii) 1.05 μg peptide/seed, or (iii) a ‘mock’ treatment that included no peptide.

3.5 inch pots were filled (approximately 0.2 L per pot) with Sunshine #1 mix (SunGro Horticulture), fertilized with 14-14-14 N,P,K Osmocote (Everris), and mixed for 20 minutes using a soil mixer M-5. Seeds were planted at a depth of 0.5 inch for germination. The pots were immediately irrigated by submergence then drained after about an hour. For each treatment group, 24 replicate seeds were planted. Plants were grown in a greenhouse under 16 hour light days at ˜75° F. and 8 hour dark nights at ˜65° F. Prior to drought conditions, the plants were well-watered.

Seven days after germination the heights of the plants were measured. This was determined as the distance between the soil surface and the shoot apical meristems (buds). The plants were randomized using a complete block design and subjected to a cyclic drought stress. Drought stress was imposed by withholding water for 3-4 days until the majority of plants were under medium to severe water stress. The severity of the stress was determined by the weight of the pot and visual determination of loss of turgor in the leaves (drooping). Once drought stress was attained, the pots were watered to full saturation. Full Saturation (100% pot capacity) is defined as the weight of the pot filled with water-saturated soil minus the weight of the filled pot prior to adding water. Drought stress was imposed for a total of 18 days.

At the end of the experiment (18 days after initiation of drought stress), the plant above the soil surface was then harvested for fresh and dry weights. Individual plants were cut near the soil surface and weighed immediately (fresh weight). The plants were then placed into labeled brown paper bags and dried in a 70° C. oven for 72 hours. A dry weight was then obtained. The following calculation was performed on the collected data: Fresh weight at harvest, and dry weight at harvest for the peptide-treated plants were expressed as a % change relative to the ‘mock’ treated plants. Results are presented in Table 9 below.

TABLE 9
Summary of Drought Resistance Following Soy Seed Treatment

| | SEQ ID | Application Rate | % Change | |
|---|---|---|---|---|
| Peptide | NO: | (μg/seed) | Dry Weight | Fresh Weight |
| P13-10 | 16 | 0.1 | −1.66 | 1.01 |
| P13-11 | 17 | 0.9 | 0.16 | 3.42 |
| P13-12 | 18 | 0.9 | −0.37 | 0.77 |
| P13-17 | 23 | 0.1 | 9.48** | 8.02* |
| P13-20 | 26 | 0.1 | −5.02 | −5.86** |
| P13-22 | 28 | 0.1 | 4.36 | 4.91 |
| P13-23 | 29 | 0.1 | 6.43 | 5.08 |
| P13-26 | 32 | 0.1 | 3.40 | 2.04 |
| P13-28 | 34 | 0.9 | 10.35** | 14.35** |
| P13-30 | 36 | 0.9 | 6.17 | 9.41* |
| P13-35 | 41 | 0.1 | 2.56 | 1.55 |
| P13-36 | 42 | 0.9 | −4.78 | 1.46 |
| P13-37 | 43 | 0.9 | 0.48 | 0.58 |
| P13-39 | 61 | 0.1 | 3.26 | 3.22 |
| P13-40 | 62 | 0.1 | 10.27** | 12.53** |
| P13-43 | 65 | 0.1 | 2.37 | 1.96 |
| P13-44 | 66 | 0.1 | 10.93** | 10.54** |
| P13-45 | 67 | 0.9 | 3.17 | 3.6 |

A select few peptides produced significant results in the seed treated drought study for soy: p13-17 (SEQ ID NO: 23), p13-28 (SEQ ID NO: 34), p13-30 (SEQ ID NO: 36), p13-40 (SEQ ID NO: 62), and p13-44 (SEQ ID NO: 66).

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

1. An isolated peptide comprising the amino acid sequence of
(SEQ ID NO: 1)
(L/M)-X-X-(L/M)-X-X-L-(L/M)-X-(L/I)-(E/L/F)-X-X-
(L/I)-X-X-X-L-(L/F)
wherein each X is independently any amino acid.

2. The isolated peptide according to claim 1, wherein each X is independently one of R, K, D, E, Q, N, H, S, T, G, P, Y, W, A, IsoD, or IsoE.

3-4. (canceled)

5. The isolated peptide according to claim 1, wherein the peptide comprises the amino acid sequence of:
(SEQ ID NO: 2)
(L/M)-X-X-(L/M)-E-(E/Q)-L-(L/M)-X-(L/I)-(E/L/F)-X-
X-(L/I)-X-(E/Q)-X-L-(L/F),
wherein each X is independently any amino acid.

6. The isolated peptide according to claim 5, wherein each X is independently one of R, K, D, E, Q, N, H, S, T, G, P, Y, W, A, IsoD, or IsoE.

7-8. (canceled)

9. The isolated peptide according to claim 5, wherein the peptide comprises the amino acid sequence of one of:

| Peptide | SEQ ID NO |
|---|---|
| QTGDDSLSGAGQTSGMSPMEQLMKIFADITQSLFGDQDG | 5, |
| GDLQGSGASTQDTSGMSPMEQLMKIFADITQSLFGDQDG | 6, |
| TSGMSPMEQLMKIFADITQSLFG | 7, |
| TSGLSPLEQLLKIFADITQSLFG | 8, |
| TSGLSPLEQLLKIFAEITQSLFG | 9, |
| MSPMEQLMKIFADITQSLFEEEE | 10, |
| LSPLEQLMKIFADITQSLFEEEE | 11, |
| MEEMEELMEIFEEIEEELFEE | 12, |
| LEELEELLEIFEEIEEELFEE | 13, |
| SEEEEMSPMEQLMKIFADITQSLF | 14, |
| SEEEEMSPMEQLMKIFAEITQSLF | 15, |
| DDSLSGAGQTSGMSPMEQLMKIFADITQSLFGDQDG | 16, |
| LSGAGQTSGMSPMEQLMKIFADITQSLFGDQDG | 17, |
| AGQTSGMSPMEQLMKIFADITQSLFGDQDG | 18, and |
| QTGDDSLSGAGQTSGMSPMEQLMKIFADITQSLFG | 20, |

10. The isolated peptide according to claim 5, wherein the peptide comprises the amino acid sequence of one of:

| Peptide | SEQ ID NO |
|---|---|
| QTGDDSLSGAGQTSGMSPMEQLMKIFADITQSLFGDQ | 19, |
| GQTSGMSPMEQLMKIFADITQSLFG | 21, |
| AGQTSGMSPMEQLMKIFADITQSLFG | 22, |
| AGQTSGMSPMEQLMKIFADITQSLFGDQ | 23, |
| AGQTSGMSPMEQLMEIFADITQSLFGDQDG | 24, |
| AGQTSGMSPMEQLMAIFADITQSLFGDQDG | 25, |
| AGQTSGMSPMEQLMEIFADITQSLFGDQDGR | 26, |
| AGQTSGMSPMEQLMAIFADITQSLFGDQDGR | 27, |
| AGQTSGMSPMEQLMEIFADITQSLFGDQDGK | 28, |
| AGQTSGLSPLEQLLKIFADITQSLFG | 29, |
| GQTSGMSPMEQLMEIFADITQSLF | 30, |
| SQTSGMSPMEQLMEIFADITQSLF | 31, |
| SQEEEMEPMEQLMEIFEEIEQELFG | 32, |
| SQEEEMEEMEQLMEIFEEIEQELFG | 33, |
| SEQEEEMEEMEQLMEIFEEIEQELFE | 34, |
| SEQEEELEELEQLLEIFEEIEQELFE | 35, |
| AGQTSGMSPMEQLMKLFADLTQSLFGDQDG | 36, |
| AGQTSGMSPMEQLMKILADITQSLFGDQDG | 37, |
| AGQTSGMSPMEQLMKIFADITQSLLGDQDG | 38, |
| AGQTSGMSPMEQLMKILADITQSLLGDQDG | 39, |
| SEQEEEMEEMEQLMEIFEEIEQELF | 40, |
| LEELEELLEIFEEIEEELF | 41, |
| SEEMSPMEQLMKIFADITQSLFEE | 42, |
| MEEMEQLMKIFEEIEQELFEEEE | 43, |
| MSPMEELMKIFADITESLFEEEE | 44, |
| MSPMEQLMKIFADITQSLFEE | 45, |
| MEEMEQLMEIFEEIEQELFEEEE | 46, |
| MSPMEQLMEIFADITQSLFEEEE | 47, |
| LEEMEELMEIFEEIEEELFEE | 48, |
| MEELEELMEIFEEIEEELFEE | 49, |
| MEEMEELLEIFEEIEEELFEE | 50, |
| MEELEELLEIFEEIEEELFEE | 51, |
| LEEMEELLEIFEEIEEELFEE | 52, and |
| LEELEELMEIFEEIEEELFEE | 53. |

11. The isolated peptide according to claim 5, wherein the peptide comprises the amino acid sequence of one of:

| Peptide | SEQ ID NO |
|---|---|
| AGQTSGMSPMEQLLKIFADITQSLFGDQDG | 61, |
| AGQTSGMSPLEQLMKIFADITQSLFGDQDG | 62, |
| AGQTSGLSPMEQLMKIFADITQSLFGDQDG | 63, |
| AGETSGMSPMEQLMKIFADITQSLFGDQDG | 64, |
| AGQTSGMSPMEQLMKIFADITESLFGDQDG | 65, |
| AGQTSGMSPMEQLMKIFADITQSLFGDEDG | 66, |
| AGQTSGMSPMEELMKIFADITQSLFGDQDG | 67, |
| AEQEEEMEPMEQLMKIFEEIEQELFEEEEE | 68, |
| AGQTSGMSPMEQLMEIFADITQSLFGDQDR | 78, |
| AGQTSGMSPMEQLMEIFADITQSLFGDQR | 79, |
| AGQTSGMSPMEQLMEIFADITQSLFGDR | 80, |
| TSGLSPLEQLLEIFADITQSLFGR | 83, and |
| TSGLSPLEQLLEIFAEITQSLFGR | 84 |

12. The isolated peptide according to claim 1, wherein the peptide comprises the amino acid sequence of:
(SEQ ID NO: 3)
(L/M)-X-X-(L/M)-E-X-L-(L/M)-X-I-F-X-X-I-X-X-X-L-F
wherein each X is independently one of R, K, D, E, Q, N, H, S, T, G, P, Y, W, or A.

13. The isolated peptide according to claim 12, wherein each X is one of E, S, P, Q, K, A, D, or T.

14. The isolated peptide according to claim 12, wherein:
X at position 2 is selected from the group consisting of E and S;
X at position 3 is selected from the group consisting of E and P;
X at position 6 is selected from the group consisting of E and Q;
X at position 9 is selected from the group consisting of E and K;
X at position 12 is selected from the group consisting of E and A;
X at position 13 is selected from the group consisting of E and D;
X at position 15 is selected from the group consisting of E and T;
X at position 16 is selected from the group consisting of E and Q; and
X at position 17 is selected from the group consisting of E and S.

15. The isolated peptide according to claim 1, wherein an arginine or lysine residue is introduced at the C-terminal end of the peptide and any lysine or arginine residues are changed to glutamate or another amino acid.

16. The isolated peptide according to claim 1, wherein the peptide comprises the amino acid sequence of:
(SEQ ID NO: 4)
T-S-G-(L/M)-S-P-(L/M)-E-Q-L-(L/M)-K-I-F-A-D-I-T-Q-
S-L-F.

17. The isolated peptide according to claim 1, wherein the peptide is up to 50 amino acids in length.

18. (canceled)

19. The isolated peptide according to claim 1, wherein the isolated peptide is stable when dissolved in water or aqueous solution, is resistant to chemical degradation when dissolved in an aqueous buffer solution containing a biocide, or has a solubility of greater than about 0.1% in water or aqueous solution.

20-21. (canceled)

22. The isolated peptide according to claim 1, wherein the peptide is at least 90% pure.

23. The isolated peptide according to claim 1, wherein the peptide is a fusion polypeptide comprising a second amino acid sequence coupled via peptide bond to the amino acid sequence.

24. The isolated peptide according to claim 23, wherein the second amino acid sequence includes a purification tag.

25. The isolated peptide according to claim 24, wherein the second amino acid sequence further includes a cleavable linker sequence between the purification tag and the amino acid sequence.

26. The isolated peptide according to claim 23, wherein the peptide is a fusion polypeptide comprising a first amino acid sequence for said peptide linked to a second amino acid sequence for said peptide.

27-28. (canceled)

29. A fusion polypeptide comprising a plurality of amino acid sequences linked together in series, each of the plurality of amino acid sequences comprising the peptide according to claim 1.

30-31. (canceled)

32. A composition comprising one or more peptides according to claim 1, and a carrier.

33. (canceled)

34. The composition according to claim 32 further comprising an additive selected from the group consisting of fertilizer, herbicide, insecticide, fungicide, nematicide, a bactericidal agent, a biological inoculant, a plant regulator, and mixtures thereof.

35-39. (canceled)

40. The composition according to claim 34, wherein the composition comprises:
one or more of peptides P12, P13-12, P13-14, P13-20, P13-3, P13-4, P13-5, P13-7, P13-s14, and P13-s15 (SEQ ID NOS: 5, 18, 20, 26, 9, 10, 11, 13, 83, and 84); and either
(i) clothianidin, a combination of clothianidin and Bacillus firmus, imidicloprid, or a combination of imidicloprid and Bacillus firmus; or
(ii) thiamethoxam; a combination of thiamethoxam, mefenoxam, and fludioxynil; a combination of thiamethoxam, mefenoxam, fludioxynil and azoxystrobin; a combination of thiamethoxam and abamectin; a combination of thiamethoxam, abamectin, and a Pasteuria nematicide; or a combination of thiamethoxam, mefenoxam, fludioxynil, azoxystrobin, thiabendazole, and abamectin; or
(iii) a biological inoculant comprising a Bradyrhizobium spp., a Bacillus spp., and a combination thereof.

41-42. (canceled)

43. The composition according to claim 32, wherein the carrier is an aqueous carrier optionally further comprising one or more of a biocidal agent, a protease inhibitor, a non-ionic surfactant, or a combination thereof.

44. (canceled)

45. The composition according to claim 32, wherein the carrier is a solid carrier in particulate form.

46. (canceled)

47. A recombinant host cell comprising a transgene that comprises a promoter-effective nucleic acid molecule operably coupled to a nucleic acid molecule that encodes a peptide according to claim 1, wherein the recombinant host cell is a microbe that imparts a first benefit to a plant grown in the presence of the recombinant microbe and the plant peptide imparts a second benefit to the plant grown in the present of the recombinant microbe.

48. The recombinant host cell according to claim 47, wherein the microbe is a bacterium or a fungus.

49-52. (canceled)

53. The recombinant host cell according to claim 47, wherein the transgene is stably integrated into the genome of the microbe.

54-56. (canceled)

57. The recombinant host cell according to claim 47, wherein the recombinant microbe is epiphytic or endophytic.

58. (canceled)

59. The recombinant host cell according to claim 47, wherein the first benefit comprises providing nutrition to a plant, producing plant hormone analogs that stimulate growth or reduce stress signaling, or competing with pathogenic organisms; and wherein the second benefit comprises disease resistance, growth enhancement, tolerance and resistance to biotic stressors, tolerance to abiotic stress, desiccation resistance for cuttings removed from ornamental plants, post-harvest disease resistance or desiccation resistance to fruit or vegetables harvested from plants, and/or improved longevity of fruit or vegetable ripeness for fruit or vegetables harvested from plants.

60. (canceled)

61. A composition comprising a plurality of recombinant host cells according to claim 47.

62-65. (canceled)

66. A mixture comprising one or more plant seeds and a composition according to claim 61.

67. A method of imparting disease resistance to plants comprising:
applying an effective amount of an isolated peptide according to claim 1 to a plant or plant seed or the locus where the plant is growing or is expected to grow, wherein said applying is effective to impart disease resistance.

68-75. (canceled)

76. A method of enhancing plant growth comprising:
applying an effective amount of an isolated peptide according to claim 1 to a plant or plant seed or the locus where the plant is growing or is expected to grow, wherein said applying is effective to enhance plant growth.

77-84. (canceled)

85. A method of increasing a plant's tolerance to biotic stress comprising:
applying an effective amount of an isolated peptide according to claim 1 to a plant or plant seed or the locus where the plant is growing or is expected to grow, wherein said applying is effective to increase the plant's tolerance to biotic stress factors selected from the group consisting of insects, arachnids, nematodes, weeds, and combinations thereof.

86-92. (canceled)

93. A method of increasing a plant's tolerance to abiotic stress comprising:
applying an effective amount of an isolated peptide according to claim 1 to a plant or plant seed or the locus where the plant is growing or is expected to grow, wherein said applying is effective to increase the plant's tolerance to abiotic stress factors selected from the group consisting of salt stress, water stress, ozone stress, heavy metal stress, cold stress, heat stress, nutritional stress, and combinations thereof.

94-121. (canceled)

122. A method for treating plant seeds comprising:
providing one or more plant seeds; and
applying to the provided one or more plant seeds either a recombinant host cell according to claim 47.

123-124. (canceled)

125. A method for treating plants comprising:
providing one or more plants; and
applying to the provided one or more plants a recombinant host cell according to claim 47.

126-127. (canceled)

128. A method for treating plants comprising:
applying to a locus where plants are being grown or are expected to be grown a recombinant host cell according to claim 47; and
growing one or more plants at the locus where the recombinant host cell is applied.

129-153. (canceled)
